SEQUENCE LISTING (28 sequences)

SEQ ID NO: 1 - protein, 509 aa, Drosophila melanogaster
MAPKSRKKKAHARSLTCYCDGSCPDNVSNGTCETRPGGSCFSAVQQLYDETTGMYEEERTYGCMPPEDNGGFLMCKVAAVPHLHGKNIVCCDKEDFCNRDLYPTYTPKLTTPAPDLPVSSESLHTLAVFGSIIISLSVFMLIVASLCFTYKRREKLRKQPRLINSMCNSQLSPLSQLVEQSSGSGSGLPLLVQRTIAKQIQMVRLVGKGRYGEVWLAKWRDERVAVKTFFTTEEASWFRETEIYQTVLMRHDNILGFIAADIKGNGSWTQMLLITDYHEMGSLHDYLSMSVINPQKLQLLAFSLASGLAHLHDEIFGTPGKPAIAHRDIKSKNILVKRNGQCAIADFGLAVKYNSELDVIHIAQNPRVGTRRYMAPEVLSQQLDPKQFEEFKRADMYSVGLVLWEMTRRCYTPVSGTKTTTCEDYALPYHDVVPSDPTFEDMHAVVCVKGFRPPIPSRWQEDDVLATVSKIMQECWHPNPTVRLTALRVKKTLGRLETDCLIDVPIKIV

SEQ ID NO: 2 - protein, 528 aa, Danio rerio
MRQLLFITVVLTGVCLLLTLCSGAGQNPDHVLQGTGVKLDSRRPGDDSTIAPEDAARFLSCHCSGHCPDDAKNNTCETNGQCFAINEEDENGDVILSSGCMKYEGSHFQCKDSQFAQTRRTIECCQFDFCNQDLKPELPPRDSEPPDPHWLAFLISVTVCFCALICVTVICYYRYKWQTERQRYHRDLEQDEAFIPAGESLKDLINQSQTSGSGSGLPLLVQRTIRKQIQTVRMIGKGRYGEVWLGRWRGEKVAVKVFFTREEASWFRETEIYQTVLMRHENILGFIAADINGTGASTQLYLITDYHENGSLYDYLKFTTLDTQALLRLAFSAACGLCHLHTEIYGTQGKPAIAHRDLKSKNILIKKNGTCCIADLGLAVKFNSDTNEVDLPLSTRMGTRRYMAPEVLDETLNKNHFQAYIMADIYSYGLVIWEMARRCVTGGIVEEYHVPYYEMVPSDPSYEDMLEVVCVKGLRPTVSNRWNSDECLRAMLKLMSECWAHNPASRLTILRVKKTLAKMVESQDIKIY

SEQ ID NO: 3 - protein, 509 aa, Homo sapiens
MVDGVMILPVLIMIALPSPSMEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLEVGLIILSVVFAVCLLACLLGVALRKFKRRNQERLNPRDVEYGTIEGLITTNVGDSTLADLLDHSCTSGSGSGLPFLVQRTVARQITLLECVGKGRYGEVWRGSWQGENVAVKIFSSRDEKSWFRETELYNTVMLRHENILGFIASDMTSRHSSTQLWLITHYHEMGSLYDYLQLTTLDTVSCLRIVLSIASGLAHLHIEIFGTQGKPAIAHRDLKSKNILVKKNGQCCIADLGLAVMHSQSTNQLDVGNNPRVGTKRYMAPEVLDETIQVDCFDSYKRVDIWAFGLVLWEVARRMVSNGIVEDYKPPFYDVVPNDPSFEDMRKVVCVDQQRPNIPNRWFSDPTLTSLAKLMKECWYQNPSARLTALRIKKTLTKIDNSLDKLKTDC

SEQ ID NO: 4 - protein, 532 aa, Homo sapiens
MPQLYIYIRLLGAYLFIISRVQGQNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRWLVLLISMAVCIIAMIIFSSCFCYKHYCKSISSRRRYNRDLEQDEAFIPVGESLKDLIDQSQSSGSGSGLPLLVQRTIAKQIQMVRQVGKGRYGEVWMGKWRGEKVAVKVFFTTEEASWFRETEIYQTVLMRHENILGFIAADIKGTGSWTQLYLITDYHENGSLYDFLKCATLDTRALLKLAYSAACGLCHLHTEIYGTQGKPAIAHRDLKSKNILIKKNGSCCIADLGLAVKFNSDTNEVDVPLNTRVGTKRYMAPEVLDESLNKNHFQPYIMADIYSFGLIIWEMARRCITGGIVEEYQLPYYNMVPSDPSYEDMREVVCVKRLRPIVSNRWNSDECLRAVLKLMSECWAHNPASRLTALRIKKTLAKMVESQDVKI

SEQ ID NO: 5 - protein, 502 aa, Homo sapiens
MLLRSAGKLNVGTKKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHRALLISVTVCSLLLVLIILFCYFRYKRQETRPRYSIGLEQDETYIPPGESLRDLIEQSQSSGSGSGLPLLVQRTIAKQIQMVKQIGKGRYGEVWMGKWRGEKVAVKVFFTTEEASWFRETEIYQTVLMRHENILGFIAADIKGTGSWTQLYLITDYHENGSLYDYLKSTTLDAKSMLKLAYSSVSGLCHLHTEIFSTQGKPAIAHRDLKSKNILVKKNGTCCIADLGLAVKFISDTNEVDIPPNTRVGTKRYMPPEVLDESLNRNHFQSYIMADMYSFGLILWEVARRCVSGGIVEEYQLPYHDLVPSDPSYEDMREIVCIKKLRPSFPNRWSSDECLRQMGKLMTECWAHNPASRLTALRVKKTLAKMSESQDIKL

SEQ ID NO: 6 - protein, 493 aa, Homo sapiens
MTRALCSALRQALLLLAAAAELSPGLKCVCLLCDSSNFTCQTEGACWASVMLTNGKEQVIKSCVSLPELNAQVFCHSSNNVTKTECCFTDFCNNITLHLPTASPNAPKLGPMELAIIITVPVCLLSIAAMLTVWACQGRQCSYRKKKRPNVEEPLSECNLVNAGKTLKDLIYDVTASGSGSGLPLLVQRTIARTIVLQEIVGKGRFGEVWHGRWCGEDVAVKIFSSRDERSWFREAEIYQTVMLRHENILGFIAADNKDNGTWTQLWLVSEYHEQGSLYDYLNRNIVTVAGMIKLALSIASGLAHLHMEIVGTQGKPAIAHRDIKSKNILVKKCETCAIADLGLAVKHDSILNTIDIPQNPKVGTKRYMAPEMLDDTMNVNIFESFKRADIYSVGLVYWEIARRCSVGGIVEEYQLPYYDMVPSDPSIEEMRKVVCDQKFRPSIPNQWQSCEALRVMGRIMRECWYANGAARLTALRIKKTISQLCVKEDCKA

SEQ ID NO: 7 - protein, 246 aa, Artificial Sequence
  Inverse Pericam core
YNSTNVYIMADKQKNGIKANFKIRHNIEDGGVQLADHYQQNTPIGDGPVLLPDNHYLSFQSALSKDPNEKRDHMVLLEFVTAAGITLGMDELYKVDGGSGGTGVSKGEELFTGVVPILVELDGDVNGHKFSVSGEGEGDATYGKLTLKLICTTGKLPVPWPTLVTTFGYGLKCFARYPDHMKQHDFFKSAMPEGYVQERTIFFKDDGNYKTRAEVKFEGDTLVNRIELKGIDFKEDGNILGHKLEY

SEQ ID NO: 8 - protein, 246 aa, Artificial Sequence
  Ratiometric Pericam core
YNSDNVYIMADKQKNGIKANFKIRHNIEDGGVQLADHYQQNTPIGDGPVLLPDNHYLSFQSALSKDPNEKRDHMVLLEFVTAAGITLGMDELYKVDGGSGGTGVSKGEELFTGVVPILVELDGDVNGHKFSVSGEGEGDATYGKLTLKLICTTGKLPVPWPTLVTTFGYGLKCFARYPDHMKQHDFFKSAMPEGYVQERTIFFKDDGNYKTRAEVKFEGDTLVNRIELKGIDFKEDGNILGHKLEY

SEQ ID NO: 9 - protein, 246 aa, Artificial Sequence
  Flash Pericam core
YNSHNVYIMADKQKNGIKANFKIRHNIEDGGVQLADHYQQNTPIGDGPVLLPDNHYLSHQSALSKDPNEKRDHMVLLEFVTAAGITLGMDELYKVDGGSGGTGVSKGEELFTGVVPILVELDGDVNGHKFSVSGEGEGDATYGKLTLKLICTTGKLPVPWPTLVTTFGYGLKCFARYPDHMKQHDFFKSAMPEGYVQERTIFFKDDGNYKTRAEVKFEGDTLVNRIELKGIDFKEDGNILGHKLEY

SEQ ID NO: 10 - protein, 246 aa, Artificial Sequence
  Pericam core
YNSHNVYIMADKQKNGIKANFKIRHNIEDGGVQLADHYQQNTPIGDGPVLLPDNHYLSYQSALSKDPNEKRDHMVLLEFVTAAGITLGMDELYKVDGGSGGTGVSKGEELFTGVVPILVELDGDVNGHKFSVSGEGEGDATYGKLTLKLICTTGKLPVPWPTLVTTFGYGLKCFARYPDHMKQHDFFKSAMPEGYVQERTIFFKDDGNYKTRAEVKFEGDTLVNRIELKGIDFKEDGNILGHKLEY

SEQ ID NO: 11 - protein, 108 aa, Drosophila melanogaster
MGVQVVPIAPGDGSTYPKNGQKVTVHYTGTLDDGTKFDSSRDRNKPFKFTIGKGEVIRGWDEGVAQLSVGQRAKLICSPDYAYGSRGHPGVIPPNSTLTFDVELLKVE

SEQ ID NO: 12 - protein, 145 aa, Homo sapiens
MGRQRAEGLGRAVEPPPGRCWSTPPVAPPARSASAAAMGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 13 - protein, 108 aa, Homo sapiens
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 14 - protein, 179 aa, Drosophila melanogaster
YSEPAFWASIAYYELNCRVGEVFHCNNNSVIVDGFTNPSNNSDRCCLGQLSNVNRNSTIENTRRHIGKGVHLYYVTGEVYAECLSDSAIFVQSRNCNYHHGFHPSTVCKIPPGCSLKIFNNQEFAQLLSQSVNNGFEAVYELTKMCTIRMSFVKGWGAEYHRQDVTSTPCWIEIHLHGP

SEQ ID NO: 15 - protein, 178 aa, Drosophila melanogaster
YHEPAFWCSISYYELNTRVGETFHASQPSITVDGFTDPSNSERFCLGLLSNVNRNEVVEQTRRHIGKGVRLYYIGGEVFAECLSDSSIFVQSPNCNQRYGWHPATVCKIPPGCNLKIFNNQEFAALLSQSVSQGFEAVYQLTRMCTIRMSFVKGWGAEYRRQTVTSTPCWIELHLNGP

SEQ ID NO: 16 - protein, 179 aa, Homo sapiens
YEEPKHWCSIVYYELNNRVGEAFHASSTSVLVDGFTDPSNNKNRFCLGLLSNVNRNSTIENTRRHIGKGVHLYYVGGEVYAECLSDSSIFVQSRNCNYHHGFHPTTVCKIPSGCSLKIFNNQEFAQLLAQSVNHGFETVYELTKMCTIRMSFVKGWGAEYHRQDVTSTPCWIEIHLHGP

SEQ ID NO: 17 - protein, 173 aa, Homo sapiens
KHWCSIVYYELNNRVGEAFHASSTSVLVDGFTDPSNNKSRFCLGLLSNVNRNSTIENTRRHIGKGVHLYYVGGEVYAECLSDSSIFVQSRNCNFHHGFHPTTVCKIPSSCSLKIFNNQEFAQLLAQSVNHGFEAVYELTKMCTIRMSFVKGWGAEYHRQDVTSTPCWIEIHLH

SEQ ID NO: 18 - protein, 178 aa, Homo sapiens
YSEPAFWCSIAYYELNQRVGETFHASQPSLTVDGFTDPSNSERFCLGLLSNVNRNATVEMTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGP

SEQ ID NO: 19 - protein, 178 aa, Homo sapiens
YCEPAFWCSISYYELNQRVGETFHASQPSMTVDGFTDPSNSERFCLGLLSNVNRNAAVELTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGP

SEQ ID NO: 20 - protein, 870 aa, Artificial Sequence
  TIPF reporter
MAPKSRKKKAHARSLTCYCDGSCPDNVSNGTCETRPGGSCFSAVQQLYDETTGMYEEERTYGCMPPEDNGGFLMCKVAAVPHLHGKNIVCCDKEDFCNRDLYPTYTPKLTTPAPDLPVSSESLHTLAVFGSIIISLSVFMLIVASLCFTYKRREKLRKQPRLINSMCNSQLSPLSQLVEQSSGSGSGLPLLVQRTIAKQIQMVRLVGKGRYGEVWLAKWRDERVAVKTFFTTEEASWFRETEIYQTVLMRHDNILGFIAADIKGNGSWTQMLLITDYHEMGSLHDYLSMSVINPQKLQLLAFSLASGLAHLHDEIFGTPGKPAIAHRDIKSKNILVKRNGQCAIADFGLAVKYNSELDVIHIAQNPRVGTRRYMAPEVLSQQLDPKQFEEFKRADMYSVGLVLWEMTRRCYTPVSGTKTTTCEDYALPYHDVVPSDPTFEDMHAVVCVKGFRPPIPSRWQEDDVLATVSKIMQECWHPNPTVRLTALRVKKTLGRLETDCLIDVPIKIVSAGYNSTNVYIMADKQKNGIKANFKIRHNIEDGGVQLADHYQQNTPIGDGPVLLPDNHYLSFQSALSKDPNEKRDHMVLLEFVTAAGITLGMDELYKVDGGSGGTGVSKGEELFTGVVPILVELDGDVNGHKFSVSGEGEGDATYGKLTLKLICTTGKLPVPWPTLVTTFGYGLKCFARYPDHMKQHDFFKSAMPEGYVQERTIFFKDDGNYKTRAEVKFEGDTLVNRIELKGIDFKEDGNILGHKLEYNGTGMGVQVVPIAPGDGSTYPKNGQKVTVHYTGTLDDGTKFDSSRDRNKPFKFTIGKGEVIRGWDEGVAQLSVGQRAKLICSPDYAYGSRGHPGVIPPNSTLTFDVELLKVE

SEQ ID NO: 21 - protein, 888 aa, Artificial Sequence
  Zebrafish ALK3 reporter
MRQLLFITVVLTGVCLLLTLCSGAGQNPDHVLQGTGVKLDSRRPGDDSTIAPEDAARFLSCHCSGHCPDDAKNNTCETNGQCFAINEEDENGDVILSSGCMKYEGSHFQCKDSQFAQTRRTIECCQFDFCNQDLKPELPPRDSEPPDPHWLAFLISVTVCFCALICVTVICYYRYKWQTERQRYHRDLEQDEAFIPAGESLKDLINQSQTSGSGSGLPLLVQRTIRKQIQTVRMIGKGRYGEVWLGRWRGEKVAVKVFFTREEASWFRETEIYQTVLMRHENILGFIAADINGTGASTQLYLITDYHENGSLYDYLKFTTLDTQALLRLAFSAACGLCHLHTEIYGTQGKPAIAHRDLKSKNILIKKNGTCCIADLGLAVKFNSDTNEVDLPLSTRMGTRRYMAPEVLDETLNKNHFQAYIMADIYSYGLVIWEMARRCVTGGIVEEYHVPYYEMVPSDPSYEDMLEVVCVKGLRPTVSNRWNSDECLRAMLKLMSECWAHNPASRLTILRVKKTLAKMVESQDIKIYAGYNSTNVYIMADKQKNGIKANFKIRHNIEDGGVQLADHYQQNTPIGDGPVLLPDNHYLSFQSALSKDPNEKRDHMVLLEFVTAAGITLGMDELYKVDGGSGGTGVSKGEELFTGVVPILVELDGDVNGHKFSVSGEGEGDATYGKLTLKLICTTGKLPVPWPTLVTTFGYGLKCFARYPDHMKQHDFFKSAMPEGYVQERTIFFKDDGNYKTRAEVKFEGDTLVNRIELKGIDFKEDGNILGHKLEYNGTGMGVQVVPIAPGDGSTYPKNGQKVTVHYTGTLDDGTKFDSSRDRNKPFKFTIGKGEVIRGWDEGVAQLSVGQRAKLICSPDYAYGSRGHPGVIPPNSTLTFDVELLKVE

SEQ ID NO: 22 - DNA, 5890 bp, Artificial Sequence
  Cloning vector
cgccattctgcctggggacgtcggagcaagcttgatttaggtgacactatagaatacaagctacttgttctttttgcaggatcccatcgattcgaattcctgcaggctacaacagcaccaacgtctatatcatggccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagcttccagtccgccctgagcaaagaccccaacgagaagcgcgatcacatggtcctgctggagttcgtgaccgccgccgggatcactctcggcatggacgagctgtacaaggtcgacggtggcagcggtggcaccggtgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctcatctgcaccaccggcaagctgcccgtgccctggcccaccctcgtgaccaccttcggctacggcctgaagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacggtaccgggatgggcgtacaagtagttccaattgctcctggtgatggcagcacctatcccaagaatggccaaaaggtcacggtccactacaccggcaccctggacgatggcaccaagttcgattcgtcgcgcgaccgcaacaagccattcaagttcaccatcggcaagggcgaggtcatccgtggctgggatgagggagttgcccagttgagcgtcggccagcgcgccaagctgatttgctcgccggactatgcctacggtagccgtggccaccccggcgtcattccgcccaactccaccctcaccttcgacgtcgagctgctcaaggtcgaataggcagctgtttgtagtctcgttttaggtttaacctaagttgcatttagttttctgtttttgttcaacttctgttgaatacgcagttccgaccttttgtccggaaattcatagttaggttctaatcgtaagttgcttgtacgtgagcggagtaaagcagaggatccttcagaggtgcctatactacggttacttaaaccgtcgcactgattgtaagctagagtttagatataagttggcaggagctagttactaaggccccttgcaaaatctttggttagaaaaatgctttaactatttgagaaatatagggagtattttggatgacacctattcatgtcgatgtacataaatatgagatcaaaacaaaaaatcaatcgaaagaatcgaaatttatatgtaacatacattaatttttgtgccagcttttgtacgctttaactaaacgagttaagtcgcccattctttgacccccgaggctttagcctcaacatagaacatgtccttatatgttttgtttgtatctagtgtttaagtgtaaactttgcagactttgtgcaagtaaatgctgccttttgtaaaatccacggaattagttgccttaagacaaagtgtaaagacaggagccaacagacaggagcccgactcccttcctccccttttagtagcacatatttttgtatttataaccagagatatatagaactggcagttgcgtatctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagtcgaccatagccaattcaatatggcgtatatggactcatgccaattcaatatggtggatctggacctgtgccaattcaatatggcgtatatggactcgtgccaattcaatatggtggatctggaccccagccaattcaatatggcggacttggcaccatgccaattcaatatggcggacttggcactgtgccaactggggaggggtctacttggcacggtgccaagtttgaggaggggtcttggccctgtgccaagtccgccatattgaattggcatggtgccaataatggcggccatattggctatatgccaggatcaatatataggcaatatccaatatggccctatgccaatatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaaggacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttagggaac

SEQ ID NO: 23 - protein, 238 aa, Aequorea victoria
MSKGEELFTGVVPILVELDGDVNGQKFSVSGEGEGDATYGKLTLKFICTTGKLPVPWPTLVTTFSYGVQCFSRYPDHMKQHDFFKSAMPEGYVQERTIFYKDDGNYKTRAEVKFEGDTLVNRIELKGIDFKEDGNILGHKMEYNYNSHNVYIMADKPKNGIKVNFKIRHNIKDGSVQLADHYQQNTPIGDGPVLLPDNHYLSTQSALSKDPNEKRDHMILLEFVTAAGITHGMDELYK

SEQ ID NO: 24 - protein, 869 aa, Artificial Sequence
  Human ALK2 reporter
MVDGVMILPVLIMIALPSPSMEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLEVGLIILSVVFAVCLLACLLGVALRKFKRRNQERLNPRDVEYGTIEGLITTNVGDSTLADLLDHSCTSGSGSGLPFLVQRTVARQITLLECVGKGRYGEVWRGSWQGENVAVKIFSSRDEKSWFRETELYNTVMLRHENILGFIASDMTSRHSSTQLWLITHYHEMGSLYDYLQLTTLDTVSCLRIVLSIASGLAHLHIEIFGTQGKPAIAHRDLKSKNILVKKNGQCCIADLGLAVMHSQSTNQLDVGNNPRVGTKRYMAPEVLDETIQVDCFDSYKRVDIWAFGLVLWEVARRMVSNGIVEDYKPPFYDVVPNDPSFEDMRKVVCVDQQRPNIPNRWFSDPTLTSLAKLMKECWYQNPSARLTALRIKKTLTKIDNSLDKLKTDCAGYNSTNVYIMADKQKNGIKANFKIRHNIEDGGVQLADHYQQNTPIGDGPVLLPDNHYLSFQSALSKDPNEKRDHMVLLEFVTAAGITLGMDELYKVDGGSGGTGVSKGEELFTGVVPILVELDGDVNGHKFSVSGEGEGDATYGKLTLKLICTTGKLPVPWPTLVTTFGYGLKCFARYPDHMKQHDFFKSAMPEGYVQERTIFFKDDGNYKTRAEVKFEGDTLVNRIELKGIDFKEDGNILGHKLEYNGTGMGVQVVPIAPGDGSTYPKNGQKVTVHYTGTLDDGTKFDSSRDRNKPFKFTIGKGEVIRGWDEGVAQLSVGQRAKLICSPDYAYGSRGHPGVIPPNSTLTFDVELLKVE

SEQ ID NO: 25 - protein, 892 aa, Artificial Sequence
  Human ALK3 reporter
MPQLYIYIRLLGAYLFIISRVQGQNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRWLVLLISMAVCIIAMIIFSSCFCYKHYCKSISSRRRYNRDLEQDEAFIPVGESLKDLIDQSQSSGSGSGLPLLVQRTIAKQIQMVRQVGKGRYGEVWMGKWRGEKVAVKVFFTTEEASWFRETEIYQTVLMRHENILGFIAADIKGTGSWTQLYLITDYHENGSLYDFLKCATLDTRALLKLAYSAACGLCHLHTEIYGTQGKPAIAHRDLKSKNILIKKNGSCCIADLGLAVKFNSDTNEVDVPLNTRVGTKRYMAPEVLDESLNKNHFQPYIMADIYSFGLIIWEMARRCITGGIVEEYQLPYYNMVPSDPSYEDMREVVCVKRLRPIVSNRWNSDECLRAVLKLMSECWAHNPASRLTALRIKKTLAKMVESQDVKIAGYNSTNVYIMADKQKNGIKANFKIRHNIEDGGVQLADHYQQNTPIGDGPVLLPDNHYLSFQSALSKDPNEKRDHMVLLEFVTAAGITLGMDELYKVDGGSGGTGVSKGEELFTGVVPILVELDGDVNGHKFSVSGEGEGDATYGKLTLKLICTTGKLPVPWPTLVTTFGYGLKCFARYPDHMKQHDFFKSAMPEGYVQERTIFFKDDGNYKTRAEVKFEGDTLVNRIELKGIDFKEDGNILGHKLEYNGTGMGVQVVPIAPGDGSTYPKNGQKVTVHYTGTLDDGTKFDSSRDRNKPFKFTIGKGEVIRGWDEGVAQLSVGQRAKLICSPDYAYGSRGHPGVIPPNSTLTFDVELLKVE

SEQ ID NO: 26 - protein, 862 aa, Artificial Sequence [sequence omitted]

SEQ ID NO: 27 - protein, 853 aa, Artificial Sequence [sequence omitted]

SEQ ID NO: 28 - protein, 856 aa, Artificial Sequence
  Alternative Human ALK6 reporter
MLLRSAGKLNVGTKKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHRALLISVTVCSLLLVLIILFCYFRYKRQETRPRYSIGLEQDETYIPPGESLRDLIEQSQSSGSGSGLPLLVQRTIAKQIQMVKQIGKGRYGEVWMGKWRGEKVAVKVFFTTEEASWFRETEIYQTVLMRHENILGFIAADIKGTGSWTQLYLITDYHENGSLYDYLKSTTLDAKSMLKLAYSSVSGLCHLHTEIFSTQGKPAIAHRDLKSKNILVKKNGTCCIADLGLAVKFISDTNEVDIPPNTRVGTKRYMPPEVLDESLNRNHFQSYIMADMYSFGLILWEVARRCVSGGIVEEYQLPYHDLVPSDPSYEDMREIVCIKKLRPSFPNRWSSDECLRQMGKLMTECWAHNPASRLTALRVKKTLAKMSESQDIKLNVYIMADKQKNGIKANFKIRHNIEDGGVQLADHYQQNTPIGDGPVLLPDNHYLSFQSALSKDPNEKRDHMVLLEFVTAAGITLGMDELYKVDGGSGGTGVSKGEELFTGVVPILVELDGDVNGHKFSVSGEGEGDATYGKLTLKLICTTGKLPVPWPTLVTTFGYGLKCFARYPDHMKQHDFFKSAMPEGYVQERTIFFKDDGNYKTRAEVKFEGDTLVNRIELKGIDFKEDGNILGHKLEYNGTGMGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

(19) United States
(12) Patent Application Publication
(10) Pub. No.: US 2010/0021931 A1
(43) Pub. Date: Jan. 28, 2010

(54) Fluorescence based reporter construct for the direct detection of TGF-beta receptor activation and modulators thereof

(75) Inventors: Christian Bökel, Dresden (DE); Thomas Weidemann, Dresden (DE)

Correspondence Address: QUINE INTELLECTUAL PROPERTY LAW GROUP, P.C., P O BOX 458, ALAMEDA, CA, 94501, US

(73) Assignee: Technische Universitat Dresden, Dresden (DE)

(21) Appl. No.: 12/459,510

(22) Filed: Jul. 2, 2009

(30) Foreign Application Priority Data
Jul. 4, 2008 (EP) 08 159 781.7

(51) Int. Cl. G01N 33/53; C07K 14/00; C12N 9/96; C07H 21/04; C12N 15/63; C12N 5/00

(52) U.S. Cl. 435/7.1; 530/402; 435/188; 536/23.4; 435/320.1; 435/325

(57) ABSTRACT

The invention comprises a fusion protein as sensor for TGF-beta receptor activity, a method for detecting receptor activity and to screening compounds for modulators of receptor activity. The fusion protein comprises a type I TGF-beta receptor, a circularly permutated fluorescent protein moiety (cpFP) and an activation state specific receptor binding domain, binding specifically to either the activated or inactive form of the TGF-beta receptor. An activation specific interaction between the receptor and the activation state specific receptor binding domain modulates the fluorescence of the cpFP inserted in between. Thus, activation of the receptor can be detected directly by a change in fluorescence of the cpFP.

### CROSS-REFERENCE TO RELATED APPLICATIONS

This application claims priority to and benefit of European Patent application EP08159781, filed, Jul. 4, 2008, the contents of which is hereby incorporated by reference for all purposes.

### FIELD OF THE INVENTION

This invention relates to a fusion protein acting as sensor for receptor activity and uses thereof, nucleic acid molecules encoding for said fusion protein, a method for detecting receptor activity and screening compounds for modulators of receptor activity using said fusion protein.

### BACKGROUND OF THE INVENTION

Sensors proteins using circularly permutated green fluorescent protein (cpGFP) as a fusion partner are known from the state of the art. For instance, WO 2005019447A discloses a monochrome fluorescent probe to measure activity of a protein phosphorylation enzyme. The monochrome fluorescent probe comprises cpGFP, a substrate domain and a phosphorylation recognition domain, whereas the substrate domain interacts with the phosphorylation recognition domain when it is phosphorylated. This probe allows measurement of a kinase activity in a cell downstream in the signalling pathway, which cannot be allocated to an activity of a specific receptor. Further the probe acts as an artificial substrate and thus competes with endogen substrates, which may lead to dominant negative effects. EP 1238982 A1 and U.S. Pat. No. 6,469,154 disclose indicators for calcium on the basis of calmodulin and fluorescent proteins.

Signalling molecules of the TGF-beta (TGFβ) family (TGF-beta, activins, bone morphogenetic proteins (BMPs) play a role in many biologically and medically relevant processes, e.g. stem cell maintenance, apoptosis regulation, inflammation processes, embryogenesis or cancer.

All TGF-beta family growth factors signal through a heterooligomeric transmembrane receptor consisting of two different Serine/Threonine kinase receptors termed type I and II receptors (Shi and Massague, 2003). After ligand binding, the type II receptor phosphorylates the type I receptor at its membrane proximal GS domain. This displaces the inhibitor FKBP12 which binds to the nonphosphorylated GS domain of the type I receptor and inactivates its kinase domain. FK506 is a FKBP-12 antagonist that prevents binding to the type I receptor (Wang, et al. 1994, Science 256:674-676).

After phosphorylation the now unmasked GS domain serves as a binding site for the R-SMAD transcription factor (in Drosophila called Mad), which is in turn phosphorylated by the type I receptor that has become active after displacement of the inhibitor. The phosphorylated R-SMAD then complexes with cofactors and enters the nucleus. Thus, phosphorylation of the type I receptor is the critical switch between the active and inactive state of the receptor (Huse et al., 2001).

This general order of events holds true for all TGF-beta growth factor subfamilies, i.e. the Bone Morphogenetic Protein (BMP) subfamily, the Activin/Nodal subtype and finally TGF-beta itself. Minor variations in signalling via the different ligand subfamilies concern the order of receptor/ligand and receptor/receptor complex formation, the presence or absence of coreceptors for ligand capture, or the recruitment of the R-Smad transcription factors to the receptor complex by cytoplasmic cofactors leading to a coupling of signalling and endocytosis. However, the actual transition from the inactive to the active state of the receptor is in all cases achieved by phosphorylation of the type I receptor GS domain.

TGF-beta signalling in vertebrates is characterized by a significant convergence of signalling processes along the pathway. In the human genome, there are 42 ORF encoding TGF-beta family growth factors that may form homo- or heterodimers with other members of their ligand subfamily. These are bound by seven type I and five type II receptors. Although these receptors show some ligand subfamily preference, there is a certain level of promiscuity, which is also reflected in the different options for receptor heterodimerization.

The R-SMAD transcription factors that bind to and become phosphorylated by the activated type I receptors exhibit a much higher receptor specificity. Thus, BMP signals are transduced by three highly related R-Smads (Smads 1, 5, and 8) and activin/TGF-beta signals by two (Smad 2 and 3). All five R-Smads bind the same Co-Smad (Smad 4) before shuttling to the nucleus and directing target gene transcription.

Current assays for TGF-beta pathway activation include immunostainings against the phosphorylated R-SMADs, the quantification of endogenous target gene expression levels, or the activity of reporter genes. However, due to the efficient nuclear import of the phospho-SMADs none of these assays impart any information about where on the surface the signal is received. In addition, they only allow discrimination between the two classes of R-Smads involved, but due to the convergence of the upstream signalling pathways they do not give any information on the receptors involved. Further, antibodies against phosphorylated R-SMAD proteins, do not allow observing receptor activity in vivo.

Stockwell, et al. (1998, Chemistry and Biology, Current Biology 5(7):385-395) measure the activation of TGF-beta receptors of type I by using a TGF-beta dependent promoter or a GFP-Smad2 fusion protein. This assay detects the activity far downstream in the TGF-beta receptor pathway, which cannot be allocated to an activity of a specific receptor.

Monitoring the activation of TGF-beta receptors directly could help to resolve the involvement of individual TGF-beta receptors and their ligands in specific signalling events.

### SUMMARY OF THE INVENTION

In various embodiments, the present invention comprises, inter alia, a fusion protein comprising a type I TGF-beta receptor, a circularly permutated fluorescent protein moiety, and an activation state specific receptor binding domain which specifically can bind to either an activated or an inactive form of the TGF-beta receptor. In various such fusion proteins, the circularly permutated fluorescent protein moiety is inserted between the C-terminus of the TGF-beta receptor and the N-terminus of the activation state specific receptor binding domain. In some embodiments, the intensity of the fluorescence of the circularly permutated fluorescent protein moiety increases upon activation of the receptor. Also, in some embodiments, the type I TGF-beta receptor can be, e.g., Activin-Activin-like Kinases (ALKs) including ACVR1B (ALK4), bone morphogenetic protein receptors (like BMPR1A (ALK3) and BMPR1B (ALK6)), TGFBR1 (ALK5), ACVR1C (ALK7), fly type I receptor BMP Thickveins (Tkv), and one or more of the sequences shown in SEQ ID No. 1 to 7, or a homolog thereof which homolog comprises a sequence identity of over 60%, over 65%, over 70%, over 75%, over 80%, over 85%, over 90%, over 95%, over 96%, over 97%, over 98%, over 99%, or over 99.5%. In some embodiments, the circularly permutated fluorescent protein is derived from an Aequorea-related fluorescent protein or a Discosoma-related fluorescent protein. In some embodiments, the circularly permutated fluorescent protein moiety can comprise, e.g., cpGFP, cpEYFP, or cpGFP or cpEYFP with one or more of the following mutations V68L, Q69K, T203H, H148D, T203F, H148T, T203F, H148D, T203F and F46L. The circularly permutated fluorescent protein can also comprise one or more of any of the sequences shown in SEQ ID No. 8 to 10, or a homolog thereof which homolog comprises a sequence identity of over 80%, over 85%, over 90%, over 95%, over 96%, over 97%, over 98%, over 99%, or over 99.5%. In some embodiments, the activation state specific receptor binding domain can comprise, e.g., FKBP-12, a Mad Homology 2 (MH2) domain of a R-SMAD, or one or more of any of the sequences shown in SEQ ID No. 11 to 19, or a homolog thereof which homolog comprises a sequence identity of over 70%, over 75%, over 80%, over 85%, over 90%, over 95%, over 96%, over 97%, over 98%, over 99%, or over 99.5%. In various embodiments, the fusion protein of the invention can comprise a sequence according to SEQ ID No. 20 or 21 or according to SEQ ID No. 24 to 28.

In other aspects, the invention comprises a nucleic acid molecule encoding a fusion protein of the invention.

In yet other aspects, the invention comprises an expression cassette or vector comprising a nucleic acid molecule that encodes a fusion protein of the invention. In some embodiments, the invention comprises a cloning cassette or vector for the construction of such an expression cassette or vector, wherein the expression cassette or vector comprises a cloning site for the insertion of a type I TGF-beta receptor coding sequence followed by a nucleic acid molecule encoding a circularly permutated fluorescent protein in frame and the coding sequence for an activation state specific receptor binding protein domain in frame and downstream of the coding sequence of the circularly permutated fluorescent protein, which binding domain specifically binds to either an activated or an inactive form of the TGF-beta receptor.

In some aspects, the invention comprises a host cell or a non human (or in some alternative embodiments, human) multicellular organism comprising a nucleic acid of the invention; comprising an expression cassette or a vector comprising such nucleic acid; comprising a cloning cassette or vector comprising such nucleic acid; and/or expressing a protein encoded by such nucleic acid.

Other aspects of the invention include kits. Such kits can comprise, e.g., a fusion protein comprising a type I TGF-beta receptor, a circularly permutated fluorescent protein moiety, and an activation state specific receptor binding domain which specifically binds to either the activated or inactive form of the TGF-beta receptor, wherein the circularly permutated fluorescent protein moiety is inserted between the C-terminus of the TGF-beta receptor and the N-terminus of the activation state specific receptor binding domain; and/or, a nucleic acid molecule encoding said fusion protein; and/or, an expression cassette or vector comprising said nucleic acid or a cloning cassette or vector comprising said nucleic acid; and/or, a host cell or multicellular organism comprising said fusion protein or said nucleic acid. Some kits can also further comprise, e.g., control reagents, buffers, or reagents for cell transfection.

Other aspects of the invention include methods for detecting TGF-beta receptor activation or detecting an effect a compound has on TGF-beta receptor activity. In various embodiments, such methods comprise: expressing a fusion protein comprising a type I TGF-beta receptor, a circularly permutated fluorescent protein moiety, and an activation state specific receptor binding domain which specifically binds to either the activated or inactive form of the TGF-beta receptor, wherein the circularly permutated fluorescent protein moiety is inserted between the C-terminus of the TGF-beta receptor and the N-terminus of the activation state specific receptor binding domain in a host cell or multi-cellular organism, excluding humans (or in some alternate embodiments including humans); and measuring the fluorescence emitted by the fusion protein. In some such methods, the method can detect activation of the receptor by exogenous ligands or ligands present in the host cell or multi-cellular organism under specific conditions. Also, in some embodiments, the methods can detect modulators of TGF-beta receptor activity wherein the method comprises activating the TGF-beta receptor and adding a compound to be tested before, in parallel, or after, activating the TGF-beta receptor and measuring changes in fluorescence emitted by the fusion protein in response to the addition of the compound to be tested. In some embodiments, the methods of the invention can detect compounds affecting signal transduction through only one or a specific group of TGF-beta receptors. In such embodiments, the method is performed with at least two different fusion proteins wherein the at least two different fusion proteins differ in their type I TGF-beta receptor component.

These and other objects and features of the invention will become more fully apparent when the following detailed description is read in conjunction with the accompanying figures.

### BRIEF DESCRIPTION OF THE FIGURES

The invention is further illustrated by, but not necessarily limited by, the following figures.

FIG. 1 illustrates the functional principle of the reporter construct according to the invention using a cpFP that is fluorescent in its free conformation and an activation state specific receptor binding domain, specifically binding to the inactive form of the TGF-beta receptor (like FKP12). FIG. 1a shows the reporter without ligand 1 bound to the surface. Activation state specific receptor binding domain 2 is bound to the intracellular GS-Domain. Circular permutated fluorescent protein (cpFP) 3 is turned into a non-fluorescent conformation. Fluorescence is OFF. FIG. 1b shows that upon ligand binding 1, the type II receptor (II) phosphorylates the GS domain of the type I receptor reporter construct (I). This displaces activation state specific receptor binding domain 2, allowing cpFP 3 to adopt a fluorescent conformation. Fluorescence is ON.

FIG. 2 shows results of injection of TIPF RNA into zebrafish embryos. Most embryos showed fluorescence restricted to the ventral side opposing the shield (FIG. 2a). FIG. 2b shows results of coinjection of BMP4 mRNA.

FIG. 3 (top) shows fluorescence of the TIPF reporter (left) that coincides with the region of increased pSmad staining (center). The right panel shows an overlay with nuclei staining using DAPI (4′, 6-Diamidino-2-phenylindol). FIG. 3 (bottom) shows that coinjection of mRNA for the extracellular BMP inhibitor Chordin abolished pathway activity and suppressed TIPF fluorescence.

FIG. 4 shows a plasmid map of a vector used herein.

FIG. 5 (panels a and b) shows the alignments of drosophila FKBP12 (query) with the corresponding human (FIG. 5a) and zebrafish (FIG. 5b) sequences (Sbjct).

### DETAILED DESCRIPTION

One of the various objectives of the present invention is to provide a construct that allows detecting TGF-beta receptor activity directly, live and in vivo with subcellular resolution. Another of the various objectives of the present invention is to provide a method to test compounds for effects on TGF-beta receptor activation and to screen compound libraries for modulators of TGF-beta receptor activation.

To solve the first objective, the invention provides a fusion protein as reporter construct to detect TGF-beta receptor activation comprising
a. a type I TGF-beta receptor,
b. a circularly permutated fluorescent protein moiety (cpFP), and
c. an activation state specific receptor binding domain, meaning a domain specifically binding to either the activated or inactive form of the TGF-beta receptor,
   wherein the circularly permutated fluorescent protein moiety is inserted between the C-terminus of the TGF-beta receptor and the N-terminus of the activation state specific receptor binding domain (as defined in c.).

The term circularly permutated fluorescent protein moiety (cpFP) stands for a protein in which the N-terminus and C-terminus of a fluorescent protein (FP) are exchanged in order and coupled via a short linker. The circularly permutated fluorescent protein moiety can adopt two conformations. One conformation is further referred to as “free” conformation, as it is the conformation wherein the cpFP spontaneously folds into, when there are no spatial restrictions. The other conformation is further referred to as “restricted” conformation as it is the conformation the cpFP folds into, when the activation state specific receptor binding domain binds to the intracellular domain of the receptor forcing the cpFP, which is inserted between them, into a different conformation. The two different conformations have different degrees of fluorescence, including no fluorescence at all. The interaction of the N- and C-terminal fusion partners modulates these fluorescence levels.

According to the invention, an activation specific interaction between the receptor and the C-terminal activation state specific receptor binding domain therefore modulates the fluorescence of the cpFP inserted in between. In consequence, the activation of the receptor leads to a change in fluorescence of the cpFP. The change in fluorescence can be either an increase or decrease. This is due to the fact that, dependent on the protein sequence of the cpFP, either the restricted or the free conformation is fluorescent. In both conformations, the overall beta barrel structure of the fluorescent protein is formed but becomes distorted by the interaction of the N- and C-terminal ends in the restricted conformation. Therefore the change from one conformation to the other does not involve a complete refolding and is consequently very fast. Preferably, the fusion protein is constructed in such a way that the intensity of the fluorescence of the circularly permutated fluorescent protein moiety increases upon activation of the receptor.

In the fusion protein according to the invention the fluorescent circularly permutated protein moiety is N-terminally linked to the C-terminus of a type I TGF-beta receptor and C-terminally linked to the N-Terminus of the activation state specific receptor binding domain.

The activation state specific receptor binding domain binds to an intracellular domain called GS domain. The GS domain is phosphorylated in the active receptor and nonphosphorylated in the inactive receptor.

In one embodiment the activation state specific receptor binding domain, preferably a FKBP12 moiety, binds exclusively to the inactive receptor, more precisely the non phosphorylated form of the GS domain. In this state the circularly permutated fluorescent protein (cpFP) is forced into a restricted conformation. Upon activation of the TGF-beta receptor, the GS domain becomes phosphorylated, and the activation state specific receptor binding domain dissociates from the receptor, which allows the circularly permutated fluorescent protein to fold back into the free conformation leading to a change in fluorescence.

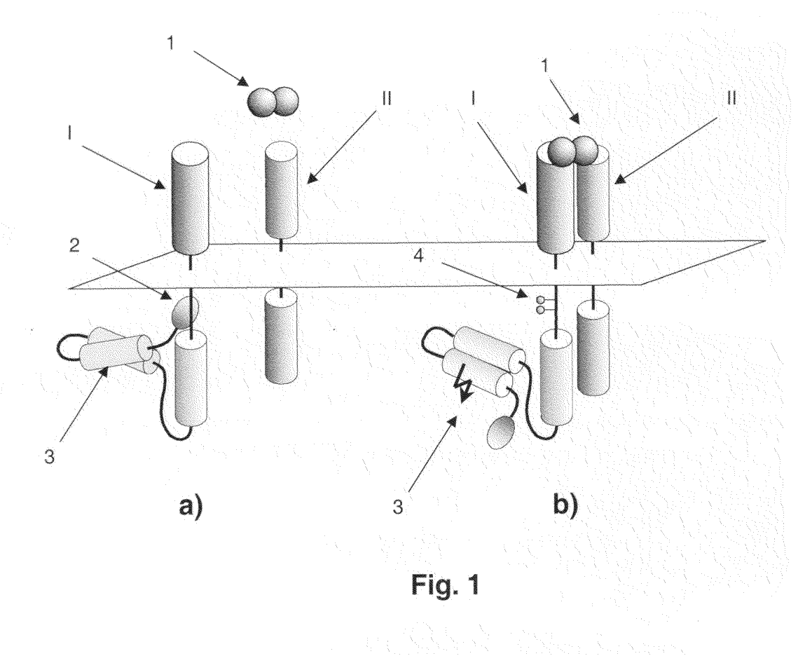

Advantageously, this insertion does not disrupt the functionality of the TGF-beta receptor: Since the FKBP12 (acting as inhibitor) is released after reporter activation just as during normal signalling, the phosphorylated binding site becomes available for recruiting the R-SMAD transcription factor that is the substrate of the type I receptor S/T kinase. Correspondingly, the inventors have verified that this reporter construct remains fully functional and that its ubiquitous expression in transgenic flies can complement the complete loss of the endogenous receptor. The principle of action of this fusion protein according to the invention is further illustrated in FIG. 1.

In an alternative embodiment, the activation state specific receptor binding domain is a domain binding exclusively to the active receptor, more precisely the phosphorylated form of the GS domain. Upon activation of the TGF-beta receptor, the GS domain becomes phosphorylated and the circularly permutated fluorescent protein (cpFP) is forced from a free into a restricted conformation leading to a change in fluorescence. In this embodiment the activation state specific receptor binding domain is preferably a Mad Homology 2 (MH2) domain of an R-SMAD. As a consequence the reporter construct in this embodiment has a dominant negative effect on the signaling pathway measured, as the binding of the MH2 domain of the reporter construct to the activated receptor prevents recruitment and activation of the naturally occurring R-SMAD.

The invention thus provides a reporter construct that allows the direct detection of the activation of the receptor. Advantageously this activation can be followed directly in a living cell or organism using a standard fluorescence microscope, and allows the detection of the subcellular location of activated receptors in the cell. As the change in fluorescence signal occurs very fast, preferably in the range of seconds, upon activation, the activation can be measured almost in real time.

In contrast to other approaches, the reporter construct according to the invention is not based on Forster-Resonance-Energie-Transfer (FRET) but uses instead only a single circularly permutated fluorescent protein. Thus, receptor activation levels can be measured directly by quantitative fluorescence microscopy without the technically challenging controls required with FRET-based systems. The reporter is therefore also useable for subcellular resolution imaging, single molecule fluorescence spectroscopy, and fluorescence recovery after photobleaching (FRAP) assays that can be used to monitor the dynamics of the activated receptors within the cell, as well as fluorescence activated cell sorting (FACS) which can be applied to distinguish and separate activated from non-activated cells.

The direct, fluorescence based detection of receptor activation is also superior to current methods for high content compound screening, as labour intensive and potentially error prone procedures such as enzyme linked immunoassays can be circumvented.

The term TGF-beta receptor according to the invention is membrane-spanning protein belonging to the TGF-beta receptor superfamily, with an extra-cellular domain binding a ligand, a trans-membrane domain and an intracellular serine/threonine kinase domain. The TGF-beta receptor according to the invention preferably binds as ligand a protein belonging to the TGF-beta family of signalling molecules. Non limiting examples of the ligands include Activins and Inhibins, Anti-Müllerian hormone (AMH), Bone morphogenetic proteins (in particular BMP2 to BMP7), Decapentaplegic protein, Growth differentiation factors (in particular GDF1 to GDF15), Nodal and Lefty, TGF-beta and isoforms of these ligands, like TGF-β1, TGF-β2 and TGF-β3.

The various fusion proteins according to the invention can contain a receptor of the TGF-beta receptor type I, which are also referred to as the Activin-Activin-like Kinases (ALKs). Preferred examples include ACVR1B (ALK4), bone morphogenetic protein receptors (like BMPR1A (ALK3) and BMPR1B (ALK6)), TGFBR1 (ALK5) and ACVR1C (ALK7), fly type I receptor BMP Thickveins (Tkv).

The TGF-beta receptor is preferably of one of the following sequences or a homologue thereof, with a sequence identity of over 60%, over 65%, preferably over 70%, over 75%, most preferably over 80%, over 85%, over 90%, over 95%, over 96%, over 97%, over 98%, over 99%, or over 99.5%.

SEQ ID No. 1 (Drosophila Tkv)
MAPKSRKKKAHARSLTCYCDGSCPDNVSNGTCETRPGGSCFSAVQQLYDE
TTGMYEEERTYGCMPPEDNGGFLMCKVAAVPHLHGKNIVCCDKEDFCNRD
LYPTYTPKLTTPAPDLPVSSESLHTLAVFGSIIISLSVFMLIVASLCFTY
KRREKLRKQPRLINSMCNSQLSPLSQLVEQSSGSGSGLPLLVQRTIAKQI
QMVRLVGKGRYGEVWLAKWRDERVAVKTFFTTEEASWFRETEIYQTVLMR
HDNILGFIAADIKGNGSWTQMLLITDYHEMGSLHDYLSMSVINPQKLQLL
AFSLASGLAHLHDEIFGTPGKPAIAHRDIKSKNILVKRNGQCAIADFGLA
VKYNSELDVIHIAQNPRVGTRRYMAPEVLSQQLDPKQFEEFKPADMYSVG
LVLWEMTRRCYTPVSGTKTTTCEDYALPYHDVVPSDPTFEDMHAVVCVKG
FRPPIPSRWQEDDVLATVSKIMQECWHPNPTVRLTALRVKKTLGRLETDC
LIDVPIKIV
SEQ ID No. 2 (Danio rerio ALK3)
MRQLLFITVVLTGVCLLLTLCSGAGQNPDHVLQGTGVKLDSRRPGDDSTI
APEDAARFLSCHCSGHCPDDAKNNTCETNGQCFAINEEDENGDVILSSGC
MKYEGSHFQCKDSQFAQTRRTIECCQFDFCNQDLKPELPPRDSEPPDPHW
LAFLISVTVCFCALICVTVICYYRYKWQTERQRYHRDLEQDEAFIPAGES
LKDLINQSQTSGSGSGLPLLVQRTIRKQIQTVRMIGKGRYGEVWLGRWRG
EKVAVKVFFTREEASWFRETEIYQTVLMRHENILGFIAADINGTGASTQL
YLITDYHENGSLYDYLKFTTLDTQALLRLAFSAACGLCHLHTEIYGTQGK
PAIAHRDLKSKNILIKKNGTCCIADLGLAVKFNSDTNEVDLPLSTRNGTR
RYNAPEVLDETLNKNHFQAYIMADIYSYGLVIWEMARRCVTGGIVEEYHV
PYYEMVPSDPSYEDMLEVVCVKGLRPTVSNRWNSDECLRANLKLMSECWA
HNPASRLTILRVKKTLAKMVESQDIKIY
SEQ ID No. 3 (Homo sapiens ALK2)
MVDGVMILPVLIMIALPSPSMEDEKPKVNPKLYMCVCEGLSCGNEDHCEG
QQCFSSLSINDGFHVYQKGCFQVYEQGKMTCKTPPSPGQAVECCQGDWCN
RNITAQLPTKGKSFPGTQNFHLEVGLIILSVVFAVCLLACLLGVALRKFK
RRNQERLNPRDVEYGTIEGLITTNVGDSTLADLLDHSCTSGSGSGLPFLV
QRTVARQITLLECVGKGRYGEVWRGSWQGENVAVKIFSSRDEKSWFRETE
LYNTVMLRHENILGFIASDMTSRHSSTQLWLITHYHEMGSLYDYLQLTTL
DTVSCLRIVLSIASGLAHLHIEIFGTQGKPAIAHRDLKSKNILVKKNGQC
CIADLGLAVMHSQSTNQLDVGNNPRVGTKRYMAPEVLDETIQVDCFDSYK
RVDIWAFGLVLWEVARRMVSNGIVEDYKPPFYDVVPNDPSFEDMRKVVCV
DQQRPNIPNRWFSDPTLTSLAKLMKECWYQNPSARLTALRIKKTLTKIDN
SLDKLKTDC
SEQ ID No. 4 (Homo sapiens ALK3)
MPQLYIYIRLLGAYLFIISRVQGQNLDSMLHGTGMKSDSDQKKSENGVTL
APEDTLPFLKCYCSGHCPDDAINNTCITNGHCFAIIEEDDQGETTLASGC
MKYEGSDFQCKDSPKAQLRRTIECCRTNLCNQYLQPTLPPVVIGPFFDGS
IRWLVLLISMAVCIIAMIIFSSCFCYKHYCKSISSRRRYNRDLEQDEAFI
PVGESLKDLIDQSQSSGSGSGLPLLVQRTIAKQIQMVRQVGKGRYGEVWM
GKWRGEKVAVKVFFTTEEASWFRETEIYQTVLMRHENILGFIAADIKGTG
SWTQLYLITDYHENGSLYDFLKCATLDTRALLKLAYSAACGLCHLHTEIY
GTQGKPAIAHRDLKSKNILIKKNGSCCIADLGLAVKFNSDTNEVDVPLNT
RVGTKRYMAPEVLDESLNKNHFQPYIMADIYSFGLIIWEMARRCITGGIV
EEYQLPYYNMVPSDPSYEDMREVVCVKRLRPIVSNRWNSDECLHAVLKLM
SECWAHNPASRLTALRIKKTLAKMVESQDVKI
SEQ ID No. 5 (Homo sapiens ALK6)
MLLRSAGKLNVGTKKEDGESTAPTPRPKVLRCKCHHHCPEDSVNNICSTD
GYCFTMIEEDDSGLPVVTSGCLGLEGSDFQCRDTPIPHQRRSIECCTERN
ECNKDLHPTLPPLKNRDFVDGPIHHRALLISVTVCSLLLVLIILFCYFRY
KRQETRPRYSIGLEQDETYIPPGESLRDLIEQSQSSCSGSGLPLLVQRTI
AKQIQMVKQIGKGRYGEVWMGKWRGEKVAVKVFFTTEEASWFRETEIYQT
VLMRHENILGFIAADIKGTGSWTQLYLITDYHENGSLYDYLKSTTLDAKS
MLKLAYSSVSGLCHLHTEIFSTQGKPAIAHRDLKSKNILVKKNGTCCIAD
LGLAVKFISDTNEVDIPPNTRVGTKRYMPPEVLDESLNRNHFQSYIMADM
YSFGLILWEVARRCVSGGIVEEYQLPYHDLVPSDPSYEDMREIVCIKKLR
PSFPNRWSSDECLRQMGKLMTECWAHNPASRLTALRVKKTLAKNSESQDI
KL
SEQ ID No. 6 (Homo sapiens ALK7)
MTRALCSALRQALLLLAAAAELSPGLKCVCLLCDSSNFTCQTEGACWASV
MLTNGKEQVIKSCVSLPELNAQVFCHSSNNVTKTECCFTDFCNNITLHLP
TASPNAPKLGPMELAIIITVPVCLLSIAAMLTVWACQGRQCSYRKKKRPN
VEEPLSECNLVNAGKTLKDLIYDVTASGSGSGLPLLVQRTIARTIVLQEI
VGKGRFGEVWHGRWCGEDVAVKIFSSRDERSWFREAEIYQTVMLRHENIL
GFIAADNKDNGTWTQLWLVSEYHEQGSLYDYLNRNIVTVAGMIKLALSIA
SGLAHLHMEIVGTQGKPAIAHRDIKSKNILVKKCETCAIADLGLAVKHDS
ILNTIDIPQNPKVGTKRYMAPEMLDDTMNVNIFESFKRADIYSVGLVYWE
IARRCSVGGIVEEYQLPYYDMVPSDPSIEEMRKVVCDQKFRPSIPNQWQS
CEALRVMGRIMRECWYANGAARLTALRIKKTISQLCVKEDCKA

In the circularly permutated fluorescent protein moiety (cpFP) the N-terminus and C-terminus of a fluorescent protein (FP) are exchanged in order and coupled via a short linker. The linker inside the cpFP is preferably between 2 and 20 amino acids long, preferably 4 to 6 amino acid residues, chosen from Glycine, Alanine and polar residues like Serine, Threonine, Glutamine or Asparagine, a particularly preferred linker being GGSGG. The fluorescent protein (FP) is preferably an Aequorea-related fluorescent protein (GFP, EGFP, YFP, EYFP, CFP, T-Sapphire and other variants thereof) or a Discosoma-related fluorescent protein (RFP and other monomeric variants of DsRed).

In various embodiments, the circularly permutated fluorescent protein moiety (cpFP) comprises preferably the following sequence in order from the N-terminus to the C-terminus:
(1) the C-terminus of the fluorescent protein (FP) or a variant thereof with a length of 105 to 115 amino acids residues,
(2) a linker sequence,
(3) the N-terminus of the fluorescent protein (FP) or a variant thereof with a length of 140 to 150 amino acids residues.

In some embodiments, this especially holds true, when the cpFP is derived from an Aequorea-related fluorescent protein (FP).

In the cpFP the amino and carboxy termini of the FP are linked by the linker peptide and thus become internal amino acids. Consequently the amino and carboxy terminal ends of the cpFP are different from the amino-terminal and carboxy-terminal amino acids of the FP.

In some embodiments, preferably the FP is an Aequorea-related fluorescent protein moiety and the cpFP comprises: the amino-terminal end of the circularly permuted Aequorea-related fluorescent protein moiety is selected from the group consisting of E142, Y143, Y145, H148, D155, H169, E172, D173, A227 and I229, and the carboxy-terminal end of the circularly permuted Aequorea-related fluorescent protein moiety is selected from the group consisting of N144, N146, N149, K162, K156, N170, I171, D173, E172, A227, and I229. The amino acid residue numbers mentioned above and below correspond to their location in the native Aequorea green fluorescent protein (SEQ ID No. 23) sequence.

In some embodiments, preferred variants of the FP are selected from the following mutations of an Aequorea-related fluorescent protein moiety: V68L, Q69K, T203H, H148D T203F, H148T, T203F, H148D, T203F and F46L.

In certain embodiments the fusion protein comprises a circularly permutated fluorescent protein (cpFP) as described in EP1238982A1, WO0071565 or U.S. Pat. No. 7,060,793B2.

Whether the “free” or the “restricted” conformation of the cpFP is more fluorescent, depends on the sequence of the cpFP. In most cases the “restricted” conformation shows higher fluorescence, which as example holds true for the sequences according to SEQ ID No. 8 to 10. These, which are preferably used when the activation state specific receptor binding domain is a domain specifically binding to the active form of the TGF-beta receptor, lead to an increase in fluorescence upon receptor activation.

In some preferred embodiments, the cpFP carries the mutations H148T, T203F, as described by Nagai, et al. (2001 PNAS 98:3197-3202) as insertion between calmodulin and M13 and called “inverse-pericam”. A modified and preferred sequence thereof is SEQ ID No. 7. This circular permutated fluorescent protein folds in its free conformation spontaneously into a fluorescent conformation. This cpFP is in particular preferred, when the activation state specific receptor binding domain is a domain specifically binding to the inactive form of the TGF-beta receptor, like FKBP12. In its restricted conformation, as long as the activation state specific receptor binding domain is bound to the receptor fluorescence is turned down or even off. Thus in this embodiment, activation of the receptor will lead to an increase in fluorescence. In this embodiment the cpFP is preferably derived from EYFP, thus cpEYFP, and preferably also carrying the mutations V68L, Q69K and F46L.

Alternatively, especially in case the circularly permutated fluorescent protein moiety (cpFP) is derived from a Discosoma-related fluorescent protein the cpFP can comprises preferably the following sequence in order from the N-terminus to the C-terminus:
(1) the C-terminus of the fluorescent protein (FP) or a variant thereof with a length of 205 to 215 amino acids residues,
(2) a linker sequence,
(3) the N-terminus of the fluorescent protein (FP) or a variant thereof with a length of 20 to 25 amino acids residues.
   or alternatively:
(1) the C-terminus of the fluorescent protein (FP) or a variant thereof with a length of 55 to 60 amino acids residues,
(2) a linker sequence,
(3) the N-terminus of the fluorescent protein (FP) or a variant thereof with a length of 180 to 190 amino acids residues.

In some embodiments, preferably the circularly permutated fluorescent protein is selected from the group consisting of cpEYFP, cpEYFP(V68L/Q69K), cpEYFP(T203H), cpEYFP(V68L/Q69K/T203H), cpEYFP(H148D/T203F), cpEYFP(V68L/Q69K/H148D/T203F), cpEYFP(H148D/T203F/F46L), cpEYFP(V68L/Q69K/H148T/T203F/F46L)), cpEYFP(H148T/T203F), cpEYFP(V68/Q69K/H148T/T203F) cpEYFP(H148T/T203F/F46L) and cpEYFP(V68L/Q69K/H148T/T203F/F46L).

The circular permutated fluorescent protein moiety can preferably be one of the following sequences or a homologue thereof, with a sequence identity of over 80%, over 85%, preferably over 90%, most preferably over 95%, over 96%, over 97%, over 98%, over 99%, or over 99.5%:

SEQ ID No. 7 (Inverse Pericam core)
YNSTNVYIMADKQKNGIKANFKIRHNIEDGGVQLADHYQQNTPIGDGPVL
LPDNHYLSFQSALSKDPNEKRDHMVLLEFVTAAGITLGMDELYKVDGGSG
GTGVSKGEELFTGVVPILVELDCDVNGHKFSVSGEGEGDATYGKLTLKLI
CTTGKLPVPWPTLVTTFGYGLKCFARYPDHMKQHDFFKSAMPEGYVQERT
IFFKDDGNYKTRAEVKFEGDTLVNRIELKGIDFKEDGNILGHKLEY
SEQ ID No. 8 (ratiometric pericam core)
YNSDNVYIMADKQKNGIKANFKIRHNIEDGGVQLADHYQQNTPIGDGPVL
LPDNHYLSFQSALSKDPNEKRDHMVLLEFVTAAGITLGMDELYKVDGGSG
GTGVSKGEELFTGVVPILVELDGDVNGHKFSVSGEGEGDATYGKLTLKLI
CTTGKLPVPWPTLVTTFGYGLKCFARYPDHMKQHDFFKSAMPEGYVQERT
IFFKDDGNYKTPAEVKFEGDTLVNRIELKGIDFKEDCNILGHKLEY
SEQ ID No. 9 (flash pericam core)
YNSHNVYIMADKQKNGIKANFKIRHNIEDGGVQLADHYQQNTPIGDGPVL
LPDNHYLSHQSALSKDPNEKRDHMVLLEFVTAAGITLGMDELYKVDGGSG
GTGVSKGEELFTGVVPILVELDGDVNGHKFSVSGEGEGDATYGKLTLKLI
CTTGKLPVPWPTLVTTFGYGLKCFARYPDHMKQHDFFKSAMPEGYVQERT
IFFKDDGNYKTRAEVKFEGDTLVNRIELKGIDFKEDGNILGHKLEY
SEQ ID No. 10 (pericam core)
YNSHNVYIMADKQKNGIKANFKIRHNIEDGGVQLADHYQQNTPIGDGPVL
LPDNHYLSYQSALSKDPNEKRDHMVLLEFVTAAGITLGMDELYKVDGGSG
GTGVSKGEELFTGVVPILVELDGDVNCHKFSVSGEGEGDATYGKLTLKLI
CTTGKLPVPWPTLVTTFGYGLKCFARYPDHMKQHDFFKSAMPEGYVQERT
IFFKDDGNYKTRAEVKFEGDTLVNRIELKGIDFKEDGNILGHKLEY

The activation state specific receptor binding domain, specifically binding to either the activated or inactive form of the TGF-beta receptor, binds preferably specifically to the intracellular GS-domain of the type I TGF-beta receptor in a activation-specific manner.

The activation state specific receptor binding domain binding specifically to the inactive form of the TGF-beta receptor is preferably the short protein called FK506-Binding Protein, 12-KD (FKBP12), also referred to as FK506-Binding Protein 1 (FKBP1), Proteinkinase C Inhibitor-2 (PKCI2) or PPIASE. The FKBP12 binds to the type I receptor GS-domain exclusively when this domain is not phosphorylated.

The activation specific binding domain binding specifically to the inactive form of the TGF-beta receptor is preferably chosen out of one of the following sequences or a homologue thereof with a sequence identity of over 70%, preferably over 75%, over 80%, most preferably over 85%, over 90%, over 95%, over 96%, over 97%, over 98%, over 99%, or over 99.5%:

SEQ ID No. 11 (Drosophila FKBPl2)
MGVQVVPIAPGDGSTYPKNGQKVTVHYTGTLDDGTKFDSSRDRNKPFKFT
IGKGEVIRGWDEGVAQLSVGQRAKLICSPDYAYGSRGHPGVIPPNSTLTF
DVELLKVE
SEQ ID NO. 12 (HUMAN FKBP1A)
MGRQRAEGLGRAVEPPPGRCWSTPPVAPPARSASAAAMGVQVETISPGDG
RTFPKRGQTCVVHYTGMLEDGKKFDSSRDRNKPFKFMLGKQEVIRGWEEG
VAQMSVGQRAKLTISPDYAYGATGHPGIIPPHATLVFDVELLKLE
SEQ ID NO. 13 (TRUNCATED HUMAN FKBP1A)
MGVQVETISPGDGRTFPKRGQTCVVHYTGMLEDGKKFDSSRDRNKPFKFM
LGKQEVIRGWEEGVAQMSVGQRAKLTISPDYAYGATGHPGIIPPHATLVF
DVELLKLE

The FKBP12 in humans and other primates is most commonly called FKBP1 and has an additional N-terminal sequence that is not essential for the interaction with the GS domain. Thus for the purposes of the invention the human and other primate sequences are preferably truncated on the N-terminus in a way that they still comprise the domain of the FKPB_C superfamily. Preferably 37 amino acid residues are removed from the N-terminus of FKBP1. A preferred example of such a truncated sequence is SEQ ID No. 13.

The conservation between insect and zebrafish FKBP12 as well as human FKBP1A is sufficiently high so that reporter constructs comprising one of these proteins can be expected to work in the other organisms as well. This has been experimentally verified for the Drosophila FKBP12 that is functional as a reporter in combination with zebrafish ALK3 (SEQ. ID No.2) in zebrafish.

Alternatively, an activation state specific receptor binding domain binding specifically to the active form of the TGF-beta receptor can be derived from the MH2 domain of the receptor type specific R-SMADs, e.g. Smad 2/3 and their homologues for activin/TGF-beta receptor signalling and SMAD 1/5/8 and their homologues for BMP receptor signalling. These domains possess a basic patch specifically binding the phosphorylated form of the receptor GS domain (Wu, et al., Science 287:92-97, January 2000) and can therefore be used to induce a closed conformation of the cpFP when the receptor is phosphorylated.

The activation specific binding domain binding specifically to the active form of the TGF-beta receptor is preferably chosen out of one of the following sequences or a homologue thereof with a sequence identity of over 70%, preferably at least 75%, over 80%, most preferably at least 85%, over 90%, over 95%, over 96%, over 97%, over 98%, over 99%, or over 99.5%:

SEQ ID NO. 14 Drosophila Mad MH2 domain
YSEPAFWASIAYYELNCRVGEVFHCNNNSVIVDGFTNPSNNSDRCCLGQL
SNVNRNSTIENTRRHIGKGVHLYYVTGEVYAECLSDSAIFVQSRNCNYHH
GFHPSTVCKIPPGCSLKIFNNQEFAQLLSQSVNNGFEAVYELTKMCTIRM
SFVKGWGAEYHRQDVTSTPCWIEIHLHGP
SEQ ID NO. 15 Drosophila Smox MH2 domain
YHEPAFWCSISYYELNTRVGETFHASQPSITVDGFTDPSNSERFCLGLLS
NVNRNEVVEQTRRHIGKGVRLYYIGGEVFAECLSDSSIFVQSPNCNQRYG
WHPATVCKIPPGCNLKIFNNQEFAALLSQSVSQGFEAVYQLTRMCTIRMS
FVKGWGAEYRRQTVTSTPCWIELHLNGP
SEQ ID NO. 16 Human Smad 1 MH2 domain
YEEPKHWCSIVYYELNNRVGEAFHASSTSVLVDGFTDPSNNKNRFCLGLL
SNVNRNSTIENTRRHIGKGVHLYYVGGEVYAECLSDSSIFVQSRNCNYHH
GFHPTTVCKIPSGCSLKIFNNQEFAQLLAQSVNHGFETVYELTKMCTIRM
SFVKGWGAEYHRQDVTSTPCWIEIHLHGP
SEQ ID NO. 17 Human Smad 5 MH2 domain
KHWCSIVYYELNNRVGEAFHASSTSVLVDGFTDPSNNKSRFCLGLLSNVN
RNSTIENTRRHIGKGVHLYYVGGEVYAECLSDSSIFVQSRNCNFHHGFHP
TTVCKIPSSCSLKIFNNQEFAQLLAQSVNHGFEAVYELTKMCTIRMSFVK
GWGAEYHRQDVTSTPCWIEIHLH
SEQ ID NO. 18 Human Smad 2 MH2 domain
YSEPAFWCSIAYYELNQRVGETFHASQPSLTVDGFTDPSNSERFCLGLLS
NVNRNATVEMTRRHIGRGVRLYYIGGEVFAECLSDSAIFVQSPNCNQRYG
WHPATVCKIPPGCNLKIFNNQEFAALLAQSVNQGFEAVYQLTRIVICTIR
NSFVKGWGAEYRRQTVTSTPCWIELHLNGP
SEQ ID NO. 19 Human Smad 3 MH2 domain
YCEPAFWCSISYYELNQRVGETFHASQPSMTVDGFTDPSNSERFCLGLLS
NVNRNAAVELTRRHIGRGVRLYYIGGEVFAECLSDSAIFVQSPNCNQRYG
WHPATVCKIPPGCNLKIFNNQEFAALLAQSVNQGFEAVYQLTRMCTIRMS
FVKGWGAEYRRQTVTSTPCWIELHLNGP

In the fusion protein according to the invention the respective sequences of the TGF-beta receptor, the cpFP and the activation state specific receptor binding domain can be bound directly to each other or linked by a sequence of 1 to 20 amino acids, preferably 1 to 6 amino acids, preferably with amino acid residues chosen from Glycine, Alanine and polar residues like Serine, Threonine, Glutamine or Asparagine. Preferred linkers are selected from the single amino acid glycine, the dipeptides AG or TG and the tripeptides TGT, NGT, TGN, TAG, SAG or GAG, as well as the tetrapeptides NGTG, TGTG, GNGT, TGNG, GTAG, GSAG or GAGT.

In some embodiments, the receptor protein and cpFP are preferably directly fused or linked by the tripeptide SAG.

In case that the activation state specific receptor binding domain is binding to the inactive receptor, like FKBP12, the cpFP and the activation state specific receptor binding domain are preferably directly fused or connected via an linker selected from the tripeptides TGT or NGT or the tetrapeptides TGTG or NGTG. In case the activation state specific receptor binding domain is binding to the active receptor, like for the MH2 domains, a longer linker, preferably with a length of 6 to 20 amino acids residues, is preferred.

Examples of the fusion protein according to the invention are given in the SEQ ID No. 20 and 21 and SEQ ID No. 24 to 28.

In another aspect, the invention provides a nucleic acid molecule, wherein the nucleic acid molecule encodes a fusion protein according to the invention. The term nucleic acid molecule does not only comprise DNA and RNA, but also nucleic acids with altered backbone, like for instance PNA. The nucleic acid molecule according to the invention comprises the coding sequence for a TGF-beta receptor including the intracellular GS domain, the coding sequence for a circularly permutated fluorescent protein in frame and downstream of the TGF-beta receptor, and the coding sequence for an activation state specific receptor binding domain of the TGF-beta receptor in frame and downstream of the circularly permutated fluorescent protein.

Further objects of the invention are an expression vector, comprising the nucleic acid molecule according to the invention, as well as a host cell or multi-cellular organism transformed with the nucleic acid molecule according to the invention. Humans are explicitly excluded from the term multi-cellular organisms in this context in some embodiments and in some embodiments of the following description. However, in some alternate embodiments herein, humans can be included within the term multi-cellular organisms.

The invention also comprises the use of a cloning cassette or vector for the construction of a nucleic acid molecule encoding a reporter construct according to the invention. This cloning cassette or vector comprises a cloning site for the insertion of a type I TGF-beta receptor coding sequence followed by a nucleic acid molecule encoding a circularly permutated fluorescent protein in frame and the coding sequence for the activation state specific receptor binding protein domain in frame and downstream of the coding sequence of the circularly permutated fluorescent protein. This cloning cassette or vector advantageously allows the insertion of a nucleic acid encoding a type I TGF-beta receptor coding sequence of interest. In this way the person applying the invention can construct a reporter construct being specific for the type I TGF-beta receptor he is interested in, e.g. to study the activation of type I TGF-beta receptor or to detect compounds activating or modulating the activity of a type I TGF-beta receptor. An example of such an expression vector is given in the SEQ ID No. 22.

Another object of the invention is a host cell or multi-cellular organism transformed with the nucleic acid molecule according to the invention. As used herein, a “host cell” is a naturally occurring cell or a transformed cell or cell line that contains an expression vector and supports the replication or expression of an expression vector. Host cells may be cultured cells, explants, cells in vivo, and the like. Host cells are preferably eukaryotic cells such as insect, amphibian, or mammalian cells such as CHO, HeLa, HEK293 and the like.

A multi-cellular organism according to the invention is a naturally occurring or otherwise genetically modified organism, preferably an invertebrate or vertebrate organism, like Drosophila melanogaster, Caenorhabditis elegans, Xenopus laevis, Medaka or Zebrafish or Mus musculus, or an embryo thereof.

The invention can also comprise transient transfectants (e.g. by mRNA), plasmid transfectants as well as host cells or multi-cellular organisms, wherein the nucleic acid molecule according to the invention is stably integrated into the genome.

The invention further comprises a kit with at least one of the following components: a fusion protein, a nucleic acid molecule, a cloning cassette or vector, an expression cassette or vector, a host cell or multicellular organism according to the invention and optionally control reagents, and buffers and/or reagents for cell transfection. In other embodiments, the kit can further comprise instructional materials (e.g., printed instructions; instructions, either printed, audio recorded or video recorded, on computer readable material such as CD or the like; indications on how to access instructions over the internet or world-wide-web, etc.) for creating and/or using the fusion proteins, etc. of the invention. Any composition, system or device of the invention can also be associated with appropriate packaging materials (e.g., containers, etc.) for production in kit form.

The invention also comprises the use of a product according to the invention, in particular the fusion protein host cell or multicellular organism or the kit (as well as the nucleic acid or the expression cassette or vector) for monitoring the activation of TGF-beta receptor or to detect compounds activating or modulating the activity of a TGF-beta receptor.

Another object of the invention is a method for detecting TGF-beta receptor activation or detecting an effect a compound has on TGF-beta receptor activity, comprising the steps of:
a.) expressing a fusion protein of the invention (e.g., a fusion protein according to one of the claims 1 to 7) in a host cell or multi-cellular organism; and
b.) measuring the fluorescence emitted by the fusion protein.

This method allows in one aspect to detect activation of the receptor by exogenous ligands or ligands present in the host cell or multi-cellular organism under specific conditions (e.g. a differentiation state, a specific cell type or cell culture conditions).

In another aspect the method is used for screening purposes and allows the screening of large compound libraries in a high content screening. The screening can be in one aspect for compounds leading to an activation of the TGF-beta receptor, by performing the following steps:
a) adding the compound or compounds to a host cell or multi-cellular organism expressing the fusion according to the invention; and
b) measuring a fluorescence emitted by the fusion protein.

A change in fluorescence compared to the non-treated control (without adding the compound to be tested) indicates an agonist or inhibitor effect on activation of the receptor. Preferably an increased fluorescence emitted by the fusion protein indicates receptor activation.

However, in some embodiments, it is even more interesting to screen for compounds modulating TGF-beta receptor activity. In this embodiment of the screening method the compound or compounds to be tested are examined for an effect on the already activated TGF-beta receptor or its activation. To detect modulators of TGF-beta receptor activity preferably a known activator of the TGF-beta receptor, like a TGF-beta growth factor, is added as additional compound preferably before or alternatively in parallel or after adding the compound to be tested. Thus the TGF-beta receptor is preferably activated by this known activator. A change in fluorescence compared to the control treated only with the known activator (without adding the compound to be tested) indicates a modulating effect, e.g. an antagonist or partial antagonist effect on activation of the receptor.

This method to detect modulators of TGF-beta receptor activation comprises preferably the following steps:
a.) providing a known activator of the TGF-beta receptor, like a TGF-beta growth factor to a host cell expressing the fusion protein according to the invention and
b.) detecting a change in said fusion protein indicating receptor activation by detecting a signal from the fluorescent protein domain and
c.) adding a compound to be tested to the host cell and quantitatively observing changes in fluorescence intensity.

As the reporter protein according to the invention allows measuring receptor activation directly, it advantageously allows screening for compounds activating a specific TGF-beta receptor.

To detect compounds affecting signal transduction only through one or a specific group of TGF-beta receptors, the method is preferably performed with at least two different fusion proteins according to the invention. In this case the fusion proteins differ at least in their TGF-beta receptor component and optionally in the colour of the cpFP.

Performing the screen with reporter constructs containing different TGF-beta type I receptor allows the identification of receptor specific compounds affecting signal transduction only through one or a specific group of receptors. This is one of the major advantages of the invention and made possible due to the fact that the invention allows the direct detection of receptor activation. A screening for such receptor specific compounds is in principle not possible using assays for TGF-beta signal transduction known from the state of the art, as they operate further down in the signalling cascade where signalling from different receptors often converges.

This screening for receptor specific compounds can be either done in parallel assays using at least two different reporter constructs according to the invention containing different TGF-beta receptors or in one host cell or organism expressing at least two different reporter constructs with cpFPs of different colours.

The compounds to be tested in the screening method of the invention can be any small chemical compound, or a protein, sugar, nucleic acid or lipid. The assays of the invention can be designed to screen large chemical libraries by high throughput screening. In some preferred embodiments, high-throughput screening methods involve providing a compound library, including but not limited to, combinatorial chemical libraries, peptide or peptide mimetic or peptoidic libraries containing a large number of potential therapeutic compounds.

### EXAMPLES

The following examples are offered to illustrate, but not to limit the claimed invention. One of skill will recognize a variety of non-critical parameters that may be altered without departing from the scope of the claimed invention.

The functionality of the fusion protein according to the invention is in the following examples illustrated by the example Drosophila Tkv reporter (further referred to as TIPF), a fusion protein consisting out of Drosophila Tkv, a TGF-beta type I receptor binding BMPs, an inverse pericam core and Drosophila FKBP12 linked by short linkers.

### Example 1

### Construction of the Drosophila Tkv Reporter (=TIPF) and mRNA Injections into Zebrafish

The protein sequence of the Drosophila Tkv reporter (further referred to as TIPF) is listed below and in SEQ ID No. 20:

MAPKSRKKKAHARSLTCYCDGSCPDNVSNGTCETRPGGSCFSAVQQLYDE
TTGMYEEERTYGCMPPEDNGCFLMCKVAAVPHLHGKNIVCCDKEDFCNRD
LYPTYTPKLTTPAPDLPVSSESLHTLAVFGSIIISLSVFMLIVASLCFTY
KRREKLRKQPRLINSMCNSQLSPLSQLVEQSSGSGSGLPLLVQRTIAKQI
QMVRLVGKGRYGEVWLAKWRDERVAVKTFFTTEEASWFRETEIYQTVLMR
HDNILGFIAADIKGNGSWTQMLLITDYHEMGSLHDYLSMSVINPQKLQLL
AFSLASGLAHLHDEIFGTPGKPAIAHRDIKSKNILVKRNGQCAIADFGLA
VKYNSELDVIHIAQNPRVGTRRYMAPEVLSQQLDPKQFEEFKRADMYSVG
LVLWEMTRRCYTPVSGTKTTTCEDYALPYHDVVPSDPTFEDMHAVVCVKG
FRPPIPSRWQEDDVLATVSKIMQECWHPNPTVRLTALRVKKTLGRLETDC
LIDVPIKIVSAGYNSTNVYIMADKQKNGIKANFKIRHNIEDGGVQLADHY
QQNTPIGDGPVLLPDNHYLSFQSALSKDPNEKRDHMVLLEFVTAAGITLG
MDELYKVDGGSGGTGVSKGEELFTGVVPILVELDGDVNGHKFSVSGEGEG
DATYGKLTLKLICTTGKLPVPWPTLVTTFGYGLKCFARYPDHMKQHDFFK
SAMPEGYVQERTIFFKDDGNYKTRAEVKFEGDTLVNRIELKGIDFKEDGN
ILGHKLEYNGTGMGVQVVPIAPGDGSTYPKNGQKVTVHYTGTLDDGTKFD
SSRDRNKPFKFTIGKGEVIRGWDEGVAQLSVGQRAKLICSPDYAYGSRGH
PGVIPPNSTLTFDVELLKVE
Underlined: Drosophila Tkv
Bold : Inverse Pericam core
Italics: Drosophila FKBP12

This reporter for signalling via receptors for the BMP-subtype of TGF-beta ligands based on the Drosophila type I BMP receptor Thickveins (Tkv) was generated as follows: A fragment of the Tkv coding sequence (CDS) encompassing the C-terminal 62 aa, the CDS of fly FKBP12, and the Tkv 3′untranslated region (UTR) were amplified in separate PCR reactions from a custom cDNA library derived from 6-12 h old Drosophila embryos. In parallel, the cpFP core domain was amplified from a plasmid containing the entire Inverse Pericam (Nagai, et al., PNAS 98 (2001), 3197-3202). Using the respective external primers for fusion PCR, the Tkv C-terminus was joined to the cpFP core and FKBP12 to the Tkv 3′UTR. Both fusion pCR products were cloned into the pCR2.1 vector (Invitrogen) and joined using the shared KpnI site at the end of the cpFP core. The entire cassette was excised with EagI and XbaI and cloned into a pCS2 expression vector containing the Tkv cDNA, replacing the C-terminus and 3′UTR with the reporter fusion. Subsequently, the entire reporter construct was excised from the expression vector and transferred into the pUAST P-element transformation vector for the generation of transgenic flies expressing the reporter under control of the compound UAS/Gal4 expression system. In a final step, the UAS site was excised and replaced by the Drosophila Ubiquitin promoter for Gal4-independent ubiquitous expression.

The functionality of the fusion protein according to the invention was tested by mRNA injections into zebrafish (Danio rerio) embryos. It is known that fly BMP receptors like TKV, which belong to the transforming growth factor beta superfamily of proteins, can bind to and be activated by vertebrate BMPs (Holley, et al., 1996). This approach is thus experimentally much faster than generating transgenic flies, which requires several generations of fly crossing per construct tested. TIPF mRNA was generated in vitro using the Message Machine kit (Ambion) using SP6 polymerase to transcribe the TIPF reporter cloned into the pCS2 expression vector.

In the early fish embryo BMP signalling drives dorsoventral patterning. Ventral expression of several activating ligands (BMP 2, 4 and 7) and dorsal expression of BMP inhibitors (Chordin, Noggin) leads to a dorsoventral BMP activity gradient (Holley and Ferguson, 1997), where high signalling levels specify ventral and low levels dorsal fates. Although molecular markers can detect that asymmetry much earlier, the first morphologically detectable marker of dorsoventral patterning is the appearance of the shield on the dorsal side after about 6 hours of development, where the internalization of future mesoderm and endoderm starts. This stage is called shield stage.

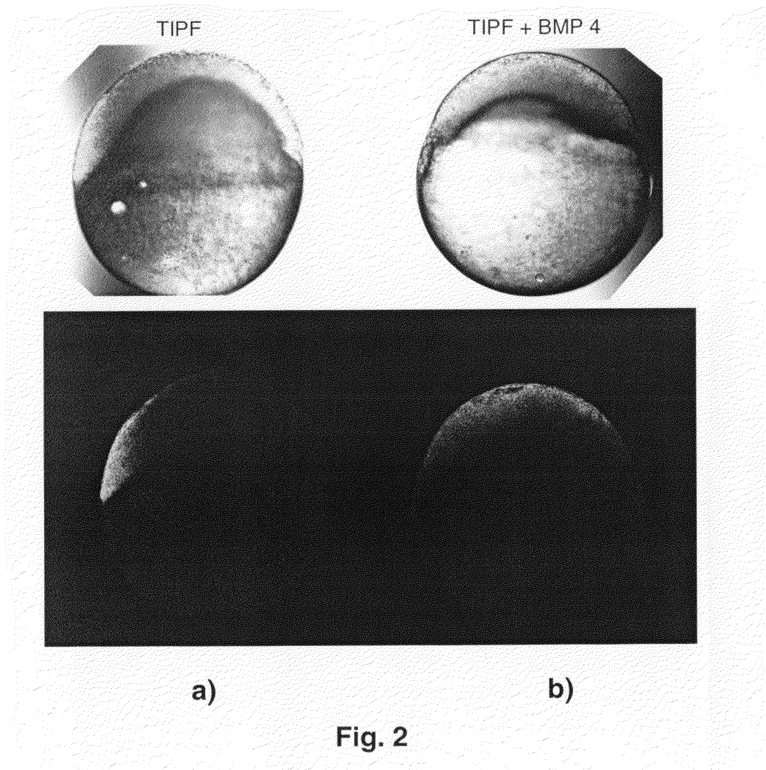

50 to 100 pg of in vitro transcribed TIPF RNA was injected into zebrafish embryos at the one cell stage, and its fluorescence activity was observed at shield stage. Indeed, most embryos showed fluorescence restricted to the ventral side opposing the shield (FIG. 2a) where BMP signalling is known to be active. Coinjection of BMP4 mRNA (100 pg/embryo) led to an expansion of the area of fluorescence (FIG. 2b) consistent with the observed ventralization of the embryo, i.e. the loss of dorsal fates that are normally specified at low levels of BMP signalling. These data suggest that the fusion protein according to the invention truthfully reflects localization of TGF-beta signalling activity.

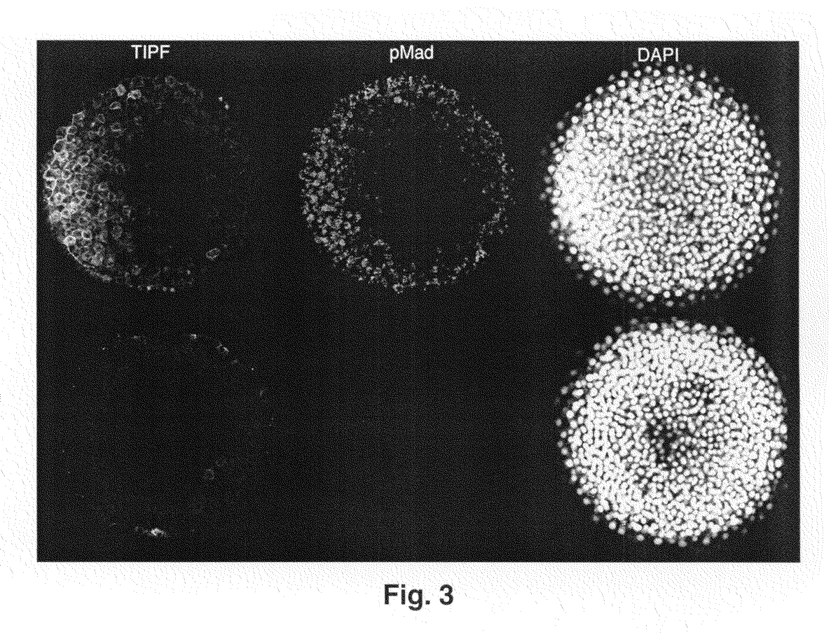

To confirm these results with molecular data, antibody stainings using a Rabbit and phospho-SMAD1/5/8 antibody (Cell Signalling Technology, 1:500) were performed on fixed Danio rerio embryos at a slightly younger stage of gastrulation (30% epiboly). While at that stage there is not yet a morphological marker for the prospective dorsal or ventral sides of the embryo, BMP pathway activation can be detected by accumulation of phosphorylated Smad1/5 transcription factors in the prospective ventral nuclei. Fluorescence of the TIPF reporter coincided with the region of BMP pathway activation as assayed by phospho-Smad immunostaining. As shown in FIG. 3 (top), fluorescence of the TIPF reporter (left) coincides with the region of increased pSmad staining (center). The right panel shows an overlay with nuclei staining using DAPI (4′,6-Diamidino-2-phenylindol).

Consistently, coinjection of mRNA for the extracellular BMP inhibitor Chordin (100 pg/embryo) both abolished pathway activity at the transcription factor level and suppressed TIPF fluorescence (FIG. 3, bottom, 30% epiboly, animal view). These data clearly show that the fusion protein according to the invention is working as a reporter for BMP receptor activation as predicted and reliably detects TGF-beta signalling activity at the receptor level.

### Example 2

### Transgenic Fly Lines Expressing the TIPF Fusion Protein

Using P-element mediated transgenesis with the plasmids described above transgenic fly lines were generated in a w¹¹¹⁸ background expressing the TIPF fusion protein under control of the ubiquitously expressed Ubiquitin promoter or in a tissue specific manner from the UAS promotor. For both transgenic construct insertions on all three major chromosomes were obtained. Insertions on the X chromosome were balanced with FM6 w⁻, 2^{nd} chromosome insertion were balanced with CyO and 3^{rd} chromosomal insertions with TM3, Sb.

The expected patterns of fluorescence could be detected e.g. in the wing disc (graded expression decaying with distance from the ligand source in the centre of the disc) in flies expressing the reporter either from the ubiquitously active ubiquitin promotor as well as from the UAS promoter in the presence of appropriate Gal4 drivers.

When expressed from the ubiquitin promoter, the reporter transgene is able to rescue the lethality associated with homozygous tkv mutants to full adult survival, demonstrating that the TIPF reporter remains a fully functional receptor.

### Example 3

### A Zebrafish ALK3-Based Fusion Protein According to the Invention

A corresponding fusion protein was made by fusing the reporter cassette consisting of the Inverse Pericam core and Drosophila FKBP12 as described in example 1 to the zebrafish BMP receptor ALK3. The protein sequence of the zebrafish ALK3 reporter is listed below and in SEQ ID No. 21.

MRQLLFITVVLTGVCLLLTLCSGAGQNPDHVLQGTGVKLDSRRPGDDSTI
APEDAARFLSCHCSGHCPDDAKNNTCETNGQCFAINEEDENGDVILSSGC
MKYEGSHFQCKDSQFAQTRRTIECCQFDFCNQDLKPELPPRDSEPPDPHW
LAFLISVTVCFCALICVTVICYYRYKWQTERQRYHRDLEQDEAFIPAGES
LKDLINQSQTSGSGSGLPLLVQRTIRKQIQTVRMIGKGRYGEVWLGRWRG
EKVAVKVFFTREEASWFRETEIYQTVLMRHENILGFIAADINGTGASTQL
YLITDYHENGSLYDYLKFTTLDTQALLRLAFSAACGLCHLHTEIYGTQGK
PAIAHRDLKSKNILIKKNGTCCIADLGLAVKFNSDTNEVDLPLSTRMGTR
RYMAPEVLDETLNKNHFQAYIMADIYSYGLVIWEMARRCVTGGIVEEYHV
PYYEMVPSDPSYEDMLEVVCVKGLRPTVSNRWNSDECLRAMLKLMSECWA
HNPASRLTILRVKKTLAKMVESQDIKIYAGYNSTNVYIMADKQKNGIKAN
FKIRHNIEDGGVQLADHYQQNTPIGDGPVLLPDNHYLSFQSALSKDPNEK
RDHMVLLEFVTAAGITLGHDELYKVDGGSGGTGVSKGEELFTGVVPILVE
LDGDVNGHKFSVSGEGEGDATYGKLTLKLICTTGKLPVPWPTLVTTFGYG
LKCFARYPDHMKQHDFFKSAMPEGYVQERTIFFKDDGNYKTRAEVKFEGD
TLVNRIELKGIDFKEDGNILGHKLEYNGTGMGVQVVPIAPGDGSTYPKNG
QKVTVHYTGTLDDGTKFDSSRDRNKPFKFTIGKGEVIRGWDEGVAQLSVG
QRAKLICSPDYAYGSRGHPGVIPPNSTLTFDVELLKVE
Underlined:
Bold: Inverse Pericam core
Itiaclics: Drosophila FKBP12

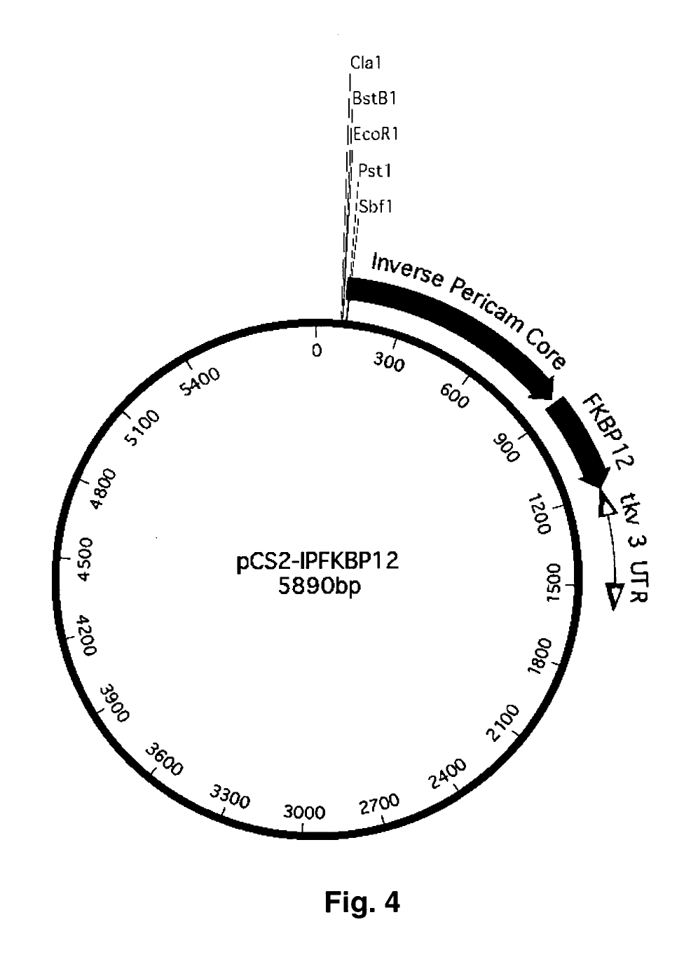

To generate the zebrafish ALK3 reporter construct, the Tkv CDS was excised from the TIPF reporter (as constructed in example 1) with PstI and EcoRI to obtain the linearized expression vector without the TGF-beta receptor according to SEQ ID No. 22. A plasmid map of this vector is given in FIG. 4. In this vector the corresponding full length cDNA encoding the zebrafish ALK3 according to SEQ ID No. 2 was cloned, which was amplified from a custom cDNA library using primers carrying EcoRI (5′primer) and NsiI (3′primer).

Zebrafish assays were performed according to example 1. In all assays this reporter behaved like the fly version used in example 1, thus demonstrating the general applicability of the detection principle.

As verified for the zebrafish reporter and supported by the blast alignments below, conservation between insect and zebrafish as well as human FKBP12 is sufficiently high so that reporters comprising one of these proteins can be expected to work in the other organisms as well.

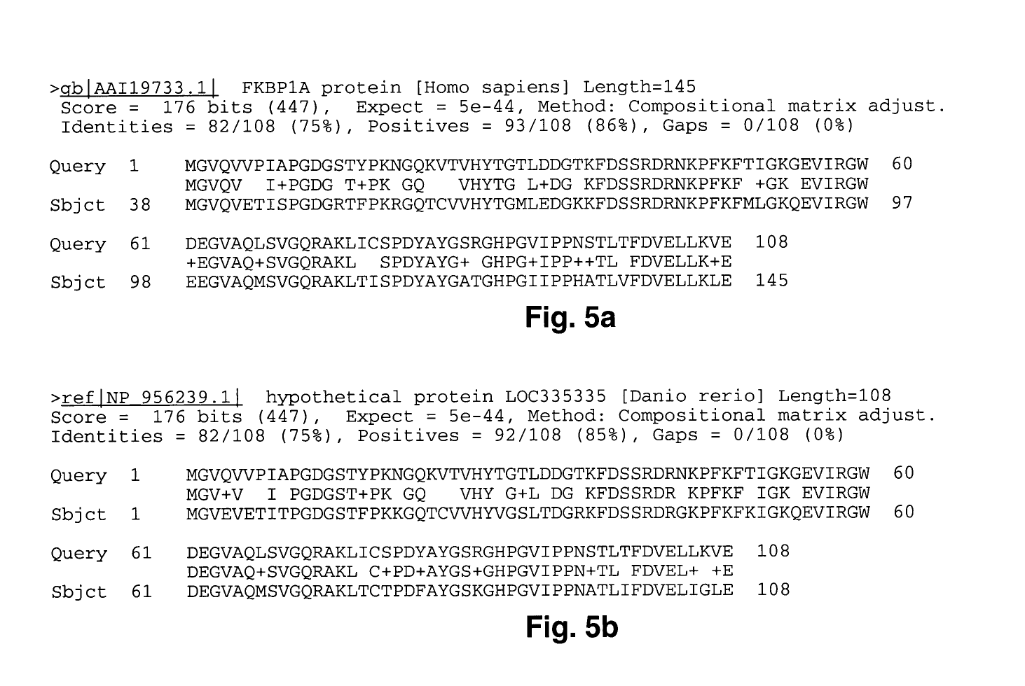

FIG. 5a and FIG. 5b show the alignments of drosophila FKBP12 (query) with the corresponding human (FIG. 5a) and zebrafish (FIG. 5b) sequences (Sbjct).

### Example 4

### Reporters for Human ALKs

Corresponding reporters for the activation of human ALKs are made by cloning a human ALK reporter, preferably encoding a sequence according to one of the SEQ ID No. 1 to 6, into the vector according to SEQ ID No. 22.

The following examples are given for human ALK reporters:

Human ALK2 reporter (SEQ ID No. 24)
MVDGVMILPVLIMIALPSPSMEDEKPKVNPKLYMCVCEGLSCGNEDHCEG
QQCFSSLSINDGFHVYQKGCFQVYEQGKMTCKTPPSPGQAVECCQGDWCN
RNITAQLPTKGKSFPGTQNFHLEVGLIILSVVFAVCLLACLLGVALRKFK
RRNQERLNPRDVEYGTIEGLITTNVGDSTLADLLDHSCTSGSGSGLPFLV
QRTVARQITLLECVGKGRYGEVWRGSWQGENVAVKIFSSRDEKSWFRETE
LYNTVMLRHENILGFIASDMTSRHSSTQLWLITHYHEMGSLYDYLQLTTL
DTVSCLRIVLSIASGLAHLHIEIFGTQGKPAIAHRDLKSKNILVKKNGQC
CIADLGLAVMHSQSTNQLDVGNNPRVGTKRYMAPEVLDETIQVDCFDSYK
RVDIWAFGLVLWEVARRMVSNGIVEDYKPPFYDVVPNDPSFEDMRKVVCV
DQQRPNIPNRWFSDPTLTSLAKLMKECWYQNPSARLTALRIKKTLTKIDN
SLDKLKTDCAGYNSTNVYIMADKQKNGIKANFKIRHNIEDGGVQLADHYQ
QNTPIGDGPVLLPDNHYLSFQSALSKDPNEKRDHMVLLEFVTAAGITLGM
DELYKVDGGSGGTGVSKGEELFTGVVPILVELDGDVNGHKFSVSGEGEGD
ATYGKLTLKLICTTGKLPVPWPTLVTTFGYGLKCFARYPDHMKQHDFFKS
AMPEGYVQERTIFFKDDGNYKTRAEVKFEGDTLVNRIELKGIDFKEDGNI
LGHKLEYNGTGMGVQVVPIAPGDGSTYPKNGQKVTVHYTGTLDDGTKFDS
SRDRNKPFKFTIGKGEVIRGWDEGVAQLSVGQRAKLICSPDYAYGSRGHP
GVIPPNSTLTFDVELLKVE
Underlined: Homo sapiens ALK2
Bold: Inverse Pericam core
Italics: Drosophila FKBP12
Human ALK3 reporter (SEQ ID No. 25)
MPQLYIYIRLLGAYLFIISRVQGQNLDSMLHGTGMKSDSDQKKSENGVTL
APEDTLPFLKCYCSGHCPDDAINNTCITNGHCFAIIEEDDQGETTLASGC
MKYEGSDFQCKDSPKAQLRRTIECCRTNLCNQYLQPTLPPVVIGPFFDGS
IRWLVLLISMAVCIIAMIIFSSCFCYKHYCKSISSRRRYNRDLEQDEAFI
PVGESLKDLIDQSQSSGSGSGLPLLVQRTIAKQIQMVRQVGKGRYGEVWM
GKWRGEKVAVKVFFTTEEASWFRETEIYQTVLMRHENILGFIAADIKGTG
SWTQLYLITDYHENGSLYDFLKCATLDTRALLKLAYSAACGLCHLHTEIY
GTQGKPAIAHRDLKSKNILIKKNGSCCIADLGLAVKFNSDTNEVDVPLNT
RVGTKRYMAPEVLDESLNKNHFQPYIMADIYSFGLIIWEMARRCITGGIV
EEYQLPYYNMVPSDPSYEDMREVVCVKRLRPIVSNRWNSDECLRAVLKLM
SECWAHNPASRLTALRIKKTLAKMVESQDVKIAGYNSTNVYIMADKQKNG
IKANFKIRHNIEDGGVQLADHYQQNTPIGDGPVLLPDNHYLSFQSALSKD
PNEKRDHMVLLEFVTAAGITLGMDELYKVDGGSGGTGVSKGEELFTGVVP
ILVELDGDVNGHKFSVSGEGEGDATYGKLTLKLICTTGKLPVPWPTLVTT
FGYGLKCFARYPDHMKQHDFFKSAMPEGYVQERTIFFKDDGNYKTRAEVK
FEGDTLVNRIELKGIDFKEDGNILGHKLEYNGTGMGVQVVPIAPGDGSTY
PKNGQKVTVHYTGTLDDGTKFDSSRDRNKPFKFTIGKGEVIRGWDEGVAQ
LSVGQRAKLICSPDYAYGSRGHPGVIPPNSTLTFDVELLKVE
Underlined: Homo sapiens ALK3
Bold : Inverse Pericam core
Italics: Drosophila FKBP12
Human ALK6 reporter (SEQ ID No. 26):
MLLRSAGKLNVGTKKEDGESTAPTPRPKVLRCKCHHHCPEDSVNNICSTD
GYCFTMIEEDDSGLPVVTSGCLGLEGSDFQCRDTPIPHQRRSIECCTERN
ECNKDLHPTLPPLKNRDFVDGPIHHRALLISVTVCSLLLVLIILFCYFRY
KRQETRPRYSIGLEQDETYIPPGESLRDLIEQSQSSGSGSGLPLLVQRTI
AKQIQMVKQIGKGRYGEVWMGKWRGEKVAVKVFFTTEEASWFRETEIYQT
VLMRHENILGFIAADIKGTGSWTQLYLITDYHENGSLYDYLKSTTLDAKS
MLKLAYSSVSGLCHLHTEIFSTQGKPAIAHRDLKSKNILVKKNGTCCIAD
LGLAVKFISDTNEVDIPPNTRVGTKRYMPPEVLDESLNRNHFQSYIMADM
YSFGLILWEVARRCVSGGIVEEYQLPYHDLVPSDPSYEDMREIVCIKKLR
PSFPNRWSSDECLRQMGKLMTECWAHNPASRLTALRVKKTLAKMSESQDI
KLAGYNSTNVYIMADKQKNGIKANFKIRHNIEDGGVQLADHYQQNTPIGD
GPVLLPDNHYLSFQSALSKDPNEKRDHMVLLEFVTAAGITLGMDELYKVD
GGSGGTGVSKGEELFTGVVPILVELDGDVNGHKFSVSGEGEGDATYGKLT
LKLICTTGKLPVPWPTLVTTFGYGLKCFARYPDHMKQHDFFKSAMPEGYV
QERTIFFKDDGNYKTRAEVKFEGDTLVNRIELKGIDFKEDGNILGHKLEY
NGTGMGVQVVPIAPGDGSTYPKNGQKVTVHYTGTLDDGTKFDSSRDRNKP
FKFTIGKGEVIRGWDEGVAQLSVGQRAKLICSPDYAYGSRGHPGVIPPNS
TLTFDVELLKVE
Underlined: Homo sapiens ALK6
Bold : Inverse Pericam core
Italics: Drosophila FKBP12
Human ALK7 reporter (SEQ ID No. 27):
MTRALCSALRQALLLLAAAAELSPGLKCVCLLCDSSNFTCQTEGACWASV
MLTNGKEQVIKSCVSLPELNAQVFCHSSNNVTKTECCFTDFCNNITLHLP
TASPNAPKLGPMELAIIITVPVCLLSIAAMLTVWACQGRQCSYRKKKRPN
VEEPLSECNLVNAGKTLKDLIYDVTASGSGSGLPLLVQRTIARTIVLQEI
VGKGRFGEVWHGRWCGEDVAVKIFSSRDERSWFREAEIYQTVMLRHENIL
GFIAADNKDNGTWTQLWLVSEYHEQGSLYDYLNRNIVTVAGMIKLALSIA
SGLAHLHMEIVGTQGKPAIAHRDIKSKNILVKKCETCAIADLGLAVKHDS
ILNTIDIPQNPKVGTKRYNAPEMLDDTMNVNIFESFKRADIYSVGLVYWE
IARRCSVGGIVEEYQLPYYDMVPSDPSIEEMRKVVCDQKFRPSIPNQWQS
CEALRVMGRIMRECWYANGAARLTALRIKKTISQLCVKEDCKAAGYNSTN
VYIMADKQKNGIKANFKIRHNIEDGGVQLADHYQQNTPIGDGPVLLPDNG
YLSFQSALSKDPNEKRDHMVLLEFVTAAGITLGMDELYKVDGGSGGTGVS
KGEELFTGVVPILVELDGDVNGHKFSVSGEGEGDATYGKLTLKLICTTGK
LPVPWPTLVTTFGYGLKCFARYPDHMKQHDFFKSAMPEGYVQERTIFFKD
DGNYKTRAEVKFEGDTLVNRIELKGIDFKEDGNILGHKLEYNGTGMGVQV
VPIAPGDGSTYPKNGQKVTVHYTGTLDDGTKFDSSRDRNKPFKFTIGKGE
VIRGWDEGVAQLSVGQRAKLICSPDYAYGSRGHPGVIPPNSTLTEDVELL
KVE
Underlined: Horno sapiens ALK7
Bold : Inverse Pericam core
Italics: Drosophila FKBP12

The Drosophila FKBP12 can, but does not have to, be replaced by a sequence encoding the truncated human FKBP1 according to SEQ ID No. 13. This is illustrated by showing a preferred protein sequence of the human ALK6 reporter listed below and in SEQ ID No. 28:

Alternative Human ALK6 reporter (SEQ ID No. 28)
MLLRSAGKLNVGTKKEDGESTAPTPRPKVLRCKCHHHCPEDSVNNICSTD
GYCFTMIEEDDSGLPVVTSGCLGLEGSDFQCRDTPIPHQRRSIECCTERN
ECNKDLHPTLPPLKNRDFVDGPIHHRALLISVTVCSLLLVLIILFCYFRY
KRQETRPRYSIGLEQDETYIPPGESLRDLIEQSQSSGSGSGLPLLVQRTI
AKQIQMVKQIGKGRYGEVWMGKWRGEKVAVKVFFTTEEASWFRETEIYQT
VLMRHENILGFIAADIKGTGSWTQLYLITDYHENGSLYDYLKSTTLDAKS
MLKLAYSSVSGLCHLHTEIFSTQGKPAIAHRDLKSKNILVKKNGTCCIAD
LGLAVKFISDTNEVDIPPNTRVGTKRYMPPEVLDESLNRNHFQSYIMADM
YSFGLILWEVARRCVSGGIVEEYQLPYHDLVPSDPSYEDMREIVCIKKLR
PSFPNRWSSDECLRQMGKLMTECWAHNPASRLTALRVKKTLAKMSESQDI
KLNVYIMADKQKNGIKANFKIRHNIEDGGVQLADHYQQNTPIGDGPVLLP
DNHYLSFQSALSKDPNEKRDHMVLLEFVTAAGITLGNDELYKVDGGSGGT
GVSKGEELFTGVVPILVELDGDVNGHKFSVSGEGEGDATYGKLTLKLICT
TGKLPVPWPTLVTTFGYGLKCFARYPDHMKQHDFFKSAMPEGYVQERTIF
FKDDGNYKTRAEVKFEGDTLVNRIELKGIDFKEDGNILGHKLEYNGTGMG
VQVETISPGDGRTFPKRGQTCVVHYTGMLEDGKKFDSSRDRNKPFKFMLG
KQEVIRGWEEGVAQMSVGQRAKLTISPDYAYGATGHPGIIPPHATLVFDV
ELLKLE
Underlined:
Bold Inverse Pericam core
Italics: truncated human FKBP1

In the same way the FKBP12 can be replaced by one of the sequences according to SEQ ID No 14 to 19.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A fusion protein comprising
a.) a type I TGF-beta receptor,
b.) a circularly permutated fluorescent protein moiety, and,
c.) an activation state specific receptor binding domain, which specifically binds to either an activated or an inactive form of the TGF-beta receptor,
wherein the circularly permutated fluorescent protein moiety is inserted between the C-terminus of the TGF-beta receptor and the N-terminus of the activation state specific receptor binding domain.

2. The fusion protein according to claim 1 wherein the intensity of the fluorescence of the circularly permutated fluorescent protein moiety increases upon activation of the receptor.

3. The fusion protein according to claim 1 wherein the type I TGF-beta receptor is selected from the group consisting of: Activin-Activin-like Kinases (ALKs), ACVR1B (ALK4), bone morphogenetic protein receptors, BMPR1A (ALK3), BMPR1B (ALK6), TGFBR1 (ALK5), ACVR1C (ALK7), fly type I receptor BMP Thickveins (Tkv), and a sequence according to any of SEQ ID No. 1 to 7 or a homolog thereof which homolog comprises a sequence identity of over 60%.

4. The fusion protein according to claim 1 wherein the circularly permutated fluorescent protein is derived from an Aequorea-related fluorescent protein or a Discosoma-related fluorescent protein.

5. The fusion protein according to claim 1 wherein the circularly permutated fluorescent protein moiety is selected from the group consisting of: cpGFP, cpEYFP, cpGFP or cpEYFP with one or more of the following mutations V68L, Q69K, T203H, H148D, T203F, H148T, T203F, H148D, T203F and F46L, and a sequence according to any of SEQ ID No. 8 to 10 or a homolog thereof which homolog comprises a sequence identity of over 80%.

6. The fusion protein according to claim 1 wherein the activation state specific receptor binding domain comprises FKBP-12, a Mad Homology 2 (MH2) domain of a R-SMAD, or a sequence according to any of SEQ ID No. 11 to 19 or a homolog thereof which homolog comprises a sequence identity of over 70%.

7. The fusion protein according to claim 1 comprising a sequence chosen from one of the sequences according to SEQ ID No. 20 and 21 and SEQ ID No. 24 to 28.

8. A nucleic acid molecule encoding a fusion protein according to claim 1.

9. An expression cassette or vector comprising a nucleic acid molecule according to claim 8.

10. A cloning cassette or vector for the construction of the expression cassette or vector according to claim 9, wherein the expression cassette or vector comprises a cloning site for the insertion of a type I TGF-beta receptor coding sequence followed by a nucleic acid molecule encoding a circularly permutated fluorescent protein in frame and the coding sequence for an activation state specific receptor binding protein domain in frame and downstream of the coding sequence of the circularly permutated fluorescent protein, which binding domain specifically binds to either an activated or an inactive form of the TGF-beta receptor.

11. A host cell or a non human multicellular organism comprising a nucleic acid according to claim 8, comprising an expression cassette or a vector comprising such nucleic acid, comprising a cloning cassette or vector comprising such nucleic acid, and/or expressing a protein encoded by such nucleic acid.

12. A Kit comprising:
a.) a fusion protein comprising a type I TGF-beta receptor, a circularly permutated fluorescent protein moiety, and an activation state specific receptor binding domain which specifically binds to either the activated or inactive form of the TGF-beta receptor, wherein the circularly permutated fluorescent protein moiety is inserted between the C-terminus of the TGF-beta receptor and the N-terminus of the activation state specific receptor binding domain; and/or,
b.) a nucleic acid molecule encoding said fusion protein; and/or,
c.) an expression cassette or vector comprising said nucleic acid or a cloning cassette or vector comprising said nucleic acid; and/or,
d.) a host cell or multicellular organism comprising said fusion protein or said nucleic acid.

13. (canceled)

14. A method for detecting TGF-beta receptor activation or detecting an effect a compound has on TGF-beta receptor activity, comprising the steps of:
a.) expressing a fusion protein comprising a type I TGF-beta receptor, a circularly permutated fluorescent protein moiety, and an activation state specific receptor binding domain which specifically binds to either the activated or inactive form of the TGF-beta receptor, wherein the circularly permutated fluorescent protein moiety is inserted between the C-terminus of the TGF-beta receptor and the N-terminus of the activation state specific receptor binding domain in a host cell or multi-cellular organism, excluding humans; and
b.) measuring the fluorescence emitted by the fusion protein

15. The method according to claim 14 wherein the method detects activation of the receptor by exogenous ligands or ligands present in the host cell or multi-cellular organism under specific conditions.

16. The method according to claim 14 wherein the method detects modulators of TGF-beta receptor activity and wherein the method comprises activating the TGF-beta receptor and adding a compound to be tested before, in parallel, or after activating the TGF-beta receptor and measuring changes in fluorescence emitted by the fusion protein in response to the addition of the compound to be tested.

17. The method according to claim 14 wherein the method detects compounds affecting signal transduction only through one or a specific group of TGF-beta receptors, the method comprising performing the method with at least two different fusion proteins, whereas wherein the at least two different fusion proteins differ in their type I TGF-beta receptor component.

18. The kit of claim 12, further comprising control reagents, buffers, or reagents for cell transfection.
